SEQUENCE LISTING (182 sequences)

SEQ ID NO: 1 - DNA, 22 bp, Homo sapiens
gggaggtggagctgccccggct

SEQ ID NO: 2 - DNA, 22 bp, Homo sapiens
ctcatccagcagcaggccgcag

SEQ ID NO: 3 - DNA, 22 bp, Homo sapiens
agcccggtattcttcctcgctg

SEQ ID NO: 4 - DNA, 22 bp, Homo sapiens
ccctccaccgggctgaccggcc

SEQ ID NO: 5 - DNA, 22 bp, Homo sapiens
gtgggccgcctactgcgcacgc

SEQ ID NO: 6 - DNA, 22 bp, Homo sapiens
ggggcccggtgtttcgcgggac

SEQ ID NO: 7 - DNA, 22 bp, Homo sapiens
gcgtcccggtgtgcgccgggcc

SEQ ID NO: 8 - DNA, 22 bp, Homo sapiens
aacgggagacctagaggggcgg

SEQ ID NO: 9 - DNA, 22 bp, Homo sapiens
ggaaaagcggtcctcggcctcc

SEQ ID NO: 10 - DNA, 22 bp, Homo sapiens
cgggtgaaaacccgacggcaac

SEQ ID NO: 11 - DNA, 22 bp, Homo sapiens
cctgcgtgtggctcctccgtgg

SEQ ID NO: 12 - DNA, 22 bp, Homo sapiens
ttgcacccttccctgcatgttt

SEQ ID NO: 13 - DNA, 22 bp, Homo sapiens
cgccggggaggcatctcctctc

SEQ ID NO: 14 - DNA, 22 bp, Homo sapiens
gaactgaacctccgtgggagtc

SEQ ID NO: 15 - DNA, 22 bp, Homo sapiens
agagagcggcttcccgttcccg

SEQ ID NO: 16 - DNA, 22 bp, Homo sapiens
ggccggctctccggacctctcc

SEQ ID NO: 17 - DNA, 22 bp, Homo sapiens
gtcgaggcctggggccggccgg

SEQ ID NO: 18 - DNA, 22 bp, Homo sapiens
gccggccccaggcctcgacgcc

SEQ ID NO: 19 - DNA, 22 bp, Homo sapiens
cctcgacgccctgggtcccttc

SEQ ID NO: 20 - DNA, 22 bp, Homo sapiens
gggggctcaccgccattcatga

SEQ ID NO: 21 - DNA, 22 bp, Homo sapiens
ggcaggcaggctccaccccttc

SEQ ID NO: 22 - DNA, 22 bp, Homo sapiens
ggccatcgcggtgagccccggc

SEQ ID NO: 23 - DNA, 22 bp, Homo sapiens
ttccgcggggagggtgctgtcc

SEQ ID NO: 24 - DNA, 22 bp, Homo sapiens
ctcgtccccgggcttccgcggg

SEQ ID NO: 25 - DNA, 22 bp, Homo sapiens
ctcgctttggctcggggtccaa

SEQ ID NO: 26 - DNA, 22 bp, Homo sapiens
aggccatcggcattccggagcc

SEQ ID NO: 27 - DNA, 22 bp, Homo sapiens
cggcgaaagcggaccgccgtca

SEQ ID NO: 28 - DNA, 22 bp, Homo sapiens
gagagacgggcctcccggagtc

SEQ ID NO: 29 - DNA, 22 bp, Homo sapiens
cctgtgcagcgcggcccccggc

SEQ ID NO: 30 - DNA, 22 bp, Homo sapiens
tgggtcgccttcgcccacaccg

SEQ ID NO: 31 - DNA, 22 bp, Homo sapiens
tttcagtttccctttacttgca

SEQ ID NO: 32 - DNA, 22 bp, Homo sapiens
tccaaaatgcaacctgaagcta

SEQ ID NO: 33 - DNA, 22 bp, Homo sapiens
cactttcaatggcgcctttttc

SEQ ID NO: 34 - DNA, 22 bp, Homo sapiens
ggaagatttctctcaaaaacgg

SEQ ID NO: 35 - DNA, 22 bp, Homo sapiens
gaggagtacgtcttctgcagcc

SEQ ID NO: 36 - DNA, 22 bp, Homo sapiens
caggtctgggctgagcattgag

SEQ ID NO: 37 - DNA, 22 bp, Homo sapiens
cctccctccagggctatggacc

SEQ ID NO: 38 - DNA, 22 bp, Homo sapiens
agctcccagcatattaggtggg

SEQ ID NO: 39 - DNA, 22 bp, Homo sapiens
gtagccctttgaccaaagggtt

SEQ ID NO: 40 - DNA, 22 bp, Homo sapiens
gcagccctgtgaccaaagggct

SEQ ID NO: 41 - DNA, 22 bp, Homo sapiens
cccagcatgcaccaggctcagg

SEQ ID NO: 42 - DNA, 22 bp, Homo sapiens
cccagcatgcaccaggctcagg

SEQ ID NO: 43 - DNA, 22 bp, Homo sapiens
gaagctcagttgtgttcacttt

SEQ ID NO: 44 - DNA, 22 bp, Homo sapiens
aactatagctatctgcaatatc

SEQ ID NO: 45 - DNA, 22 bp, Homo sapiens
atacgatccagcaatttcacat

SEQ ID NO: 46 - DNA, 21 bp, Homo sapiens
gtcaagcagatgccaagtcgt

SEQ ID NO: 47 - DNA, 21 bp, Homo sapiens
tcagtaaatgttctgtcattg

SEQ ID NO: 48 - DNA, 21 bp, Homo sapiens
atgagccaagtgaaaacacca

SEQ ID NO: 49 - DNA, 21 bp, Homo sapiens
ggcatcacaaatggcaaacca

SEQ ID NO: 50 - DNA, 21 bp, Homo sapiens
aatgcataaagcctgaaggga

SEQ ID NO: 51 - DNA, 21 bp, Homo sapiens
gaacggacagcaaacgacaag

SEQ ID NO: 52 - DNA, 21 bp, Homo sapiens
atcgataaatacgctaaccac

SEQ ID NO: 53 - DNA, 21 bp, Homo sapiens
gtcacccgcattctttagaga

SEQ ID NO: 54 - DNA, 21 bp, Homo sapiens
gaatattttgaaaattaagtg

SEQ ID NO: 55 - DNA, 21 bp, Homo sapiens
tgctggagaaaccctctttat

SEQ ID NO: 56 - DNA, 21 bp, Homo sapiens
tagcatcttaaatctccaccc

SEQ ID NO: 57 - DNA, 21 bp, Homo sapiens
aatcaaaatgcacatgctctg

SEQ ID NO: 58 - DNA, 21 bp, Homo sapiens
acttttaagacgtggagcact

SEQ ID NO: 59 - DNA, 21 bp, Homo sapiens
ttttggagcttgtccacttaa

SEQ ID NO: 60 - DNA, 21 bp, Homo sapiens
acaagactcacgcctcagaaa

SEQ ID NO: 61 - DNA, 21 bp, Homo sapiens
cgtactcctctggaaagaacc

SEQ ID NO: 62 - DNA, 21 bp, Homo sapiens
ggaaaaggtaaggtatctttg

SEQ ID NO: 63 - DNA, 21 bp, Homo sapiens
tgccgactctttgcaaggaga

SEQ ID NO: 64 - DNA, 21 bp, Homo sapiens
agctataatcccagcatgtac

SEQ ID NO: 65 - DNA, 21 bp, Homo sapiens
gcgcagccctggaagaagggg

SEQ ID NO: 66 - DNA, 21 bp, Homo sapiens
caaagggttgggagtgtttat

SEQ ID NO: 67 - DNA, 21 bp, Homo sapiens
gcaagttgaaagaaggtccct

SEQ ID NO: 68 - DNA, 21 bp, Homo sapiens
gtagttcttaggagctagagg

SEQ ID NO: 69 - DNA, 21 bp, Homo sapiens
tcattccacttatgagatact

SEQ ID NO: 70 - DNA, 21 bp, Homo sapiens
catgcatgacgtaccatgtgt

SEQ ID NO: 71 - DNA, 21 bp, Homo sapiens
tgatggtaattcaaacaacac

SEQ ID NO: 72 - DNA, 21 bp, Homo sapiens
acatttccaccaacagtgtac

SEQ ID NO: 73 - DNA, 21 bp, Homo sapiens
ggtattaagtgatatgtcatt

SEQ ID NO: 74 - DNA, 21 bp, Homo sapiens
aagtagtttgttttattgttg

SEQ ID NO: 75 - DNA, 21 bp, Homo sapiens
taggggaaacacgtcataaca

SEQ ID NO: 76 - DNA, 21 bp, Homo sapiens
ctgtaagaaaaaattaactaa

SEQ ID NO: 77 - DNA, 20 bp, Artificial Sequence
  control non-targeting targeting sequence
acggaggctaagcgtcgcaa

SEQ ID NO: 78 - DNA, 20 bp, Artificial Sequence
  control non-targeting targeting sequence
cgcttccgcggcccgttcaa

SEQ ID NO: 79 - DNA, 20 bp, Artificial Sequence
  control non-targeting targeting sequence
gtaggcgcgccgctctctac

SEQ ID NO: 80 - RNA, 19 nt, Francisella novicid
  misc_feature (1)..(19): 5′-handle of crRNA
aauuucuacuguuguagau

SEQ ID NO: 81 - protein, 1053 aa, Staphylococcus aureus
  VARIANT (10)..(10): conversion of Asp residue into Ala residue
  VARIANT (580)..(580): conversion of Asn residue into Ala residue
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 82 - protein, 1053 aa, Staphylococcus aureus
  VARIANT (10)..(10): conversion of Asp residue into Ala residue
  VARIANT (557)..(557): conversion of His residue into Ala residue
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDAIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 83 - protein, 1028 aa, Artificial Sequence
  amino acid residues  (721st to 745th amino acid residues of dSaCas9) deletion mutant
  VARIANT (10)..(10): conversion of Asp residue into Ala residue
  VARIANT (580)..(580): conversion of Asn residue into Ala residue
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKTEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 84 - protein, 6 aa, Artificial Sequence
  GGSGGS linker
GGSGGS

SEQ ID NO: 85 - protein, 1034 aa, Artificial Sequence
  amino acid residues  (721st to 745th amino acid residues of dSaCas9) deletion mutant with GGSGGS linker
  VARIANT (10)..(10): conversion of Asp residue into Ala residue
  VARIANT (580)..(580): conversion of Asn residue into Ala residue
  MISC_FEATURE (721)..(726): GGSGGS linker
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKGGSGGSTEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 86 - protein, 5 aa, Artificial Sequence
  SGGGS linker
SGGGS

SEQ ID NO: 87 - protein, 1033 aa, Artificial Sequence
  amino acid residues  (721st to 745th amino acid residues of dSaCas9) deletion mutant with SGGGS linker
  VARIANT (10)..(10): conversion of Asp residue into Ala residue
  VARIANT (580)..(580): conversion of Asn residue into Ala residue
  MISC_FEATURE (721)..(725): SGGGS linker
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKSGGGSTEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 88 - protein, 886 aa, Artificial Sequence
  amino acid residues  (482nd to 648th amino acid residues of dSaCas9) deletion mutant
  VARIANT (10)..(10): conversion of Asp residue into Ala residue
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELARETRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 89 - protein, 892 aa, Artificial Sequence
  amino acid residues  (482nd to 648th amino acid residues of dSaCas9) deletion mutant with GGSGGS linker
  VARIANT (10)..(10): conversion of Asp residue into Ala residue
  MISC_FEATURE (482)..(487): GGSGGS linker
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREGGSGGSTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 90 - DNA, 48 bp, Artificial Sequence
  DNA sequence encoding NLS
gccccaaagaagaagcggaaggtcggtatccacggagtcccagcagcc

SEQ ID NO: 91 - DNA, 48 bp, Artificial Sequence
  DNA sequence encoding NLS
aaaaggccggcggccacgaaaaaggccggccaggcaaaaaagaaaaag

SEQ ID NO: 92 - DNA, 3477 bp, Artificial Sequence
  dSaCas9 fused to KRAB (DNA)
atggccccaaagaagaagcggaaggtcggtatccacggagtcccagcagccaagcggaactacatcctgggcctggccatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaagccagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccatggatgctaagtcactaactgcctggtcccggacactggtgaccttcaaggatgtatttgtggacttcaccagggaggagtggaagctgctggacactgctcagcagatcgtgtacagaaatgtgatgctggagaactataagaacctggtttccttgggttatcagcttactaagccagatgtgatcctccggttggagaagggagaagagccc

SEQ ID NO: 93 - RNA, 22 nt, Homo sapiens
  misc_feature (1)..(22): crRNA corresponding to the target sequence (SEQ ID NO4)
cccuccaccgggcugaccggcc

SEQ ID NO: 94 - DNA, 22 bp, Homo sapiens
  misc_feature (1)..(22): sequence complementary to the target sequence (SEQ ID NO4)
ggccggtcagcccggtggaggg

SEQ ID NO: 95 - DNA, 19 bp, Francisella novicid
  misc_feature: 5′-handle of crRNA
aatttctactgttgtagat

SEQ ID NO: 96 - DNA, 60 bp, Staphylococcus aureus
  misc_feature (1)..(83): sequence encoding tracrRNA
gttttagtactctggaaacagaatctactaaaacaaggcaaaatgccgtgtttatctcgt

SEQ ID NO: 97 - DNA, 82 bp, Staphylococcus aureus
  misc_feature (1)..(82): tracRNA
gttttagtactctggaaacagaatctactaaaacaaggcaaaatgccgtgtttatctcgtcaacttgttggcgagatttttt

SEQ ID NO: 98 - DNA, 22 bp, Homo sapiens
ctaaggctttttctctccctcc

SEQ ID NO: 99 - DNA, 22 bp, Homo sapiens
tgggaggattttgcctgtgagt

SEQ ID NO: 100 - DNA, 22 bp, Homo sapiens
agattctgggaggattttgcct

SEQ ID NO: 101 - DNA, 22 bp, Homo sapiens
gaactcacaggcaaaatcctcc

SEQ ID NO: 102 - DNA, 22 bp, Homo sapiens
ttatgttctcacaagattctgg

SEQ ID NO: 103 - DNA, 22 bp, Homo sapiens
tgactagtttggcattgctttt

SEQ ID NO: 104 - DNA, 22 bp, Homo sapiens
gatttataaataatggcatgac

SEQ ID NO: 105 - DNA, 22 bp, Homo sapiens
gtgaggggagatggggagacat

SEQ ID NO: 106 - DNA, 22 bp, Homo sapiens
ccagccaggccgcgccggcaga

SEQ ID NO: 107 - DNA, 22 bp, Homo sapiens
ctcccaacctgccccggcgcgc

SEQ ID NO: 108 - DNA, 22 bp, Homo sapiens
tgcggaggccaccgaggagcct

SEQ ID NO: 109 - DNA, 22 bp, Homo sapiens
tcccggtcctcccggcttttgc

SEQ ID NO: 110 - DNA, 22 bp, Homo sapiens
gggcccggcaggccgtcgcgct

SEQ ID NO: 111 - DNA, 22 bp, Homo sapiens
ctcaagcggggccgcagggcca

SEQ ID NO: 112 - DNA, 22 bp, Homo sapiens
ccaccacggactcccctgggac

SEQ ID NO: 113 - DNA, 22 bp, Homo sapiens
gcttgcgccacccacgtcccag

SEQ ID NO: 114 - DNA, 22 bp, Homo sapiens
gagtccgtggtggggctggggc

SEQ ID NO: 115 - DNA, 22 bp, Homo sapiens
ctgctggaggagctttaggacg

SEQ ID NO: 116 - DNA, 22 bp, Homo sapiens
gctttaggacgcggggttggga

SEQ ID NO: 117 - DNA, 22 bp, Homo sapiens
aggacgcggggttgggacgggg

SEQ ID NO: 118 - DNA, 22 bp, Homo sapiens
caccggcctggacctagaaggc

SEQ ID NO: 119 - DNA, 22 bp, Homo sapiens
agaatggcagttctccgcggtg

SEQ ID NO: 120 - DNA, 22 bp, Homo sapiens
gggatccccgggatgcccagga

SEQ ID NO: 121 - DNA, 22 bp, Homo sapiens
gccattctttcctgggcatccc

SEQ ID NO: 122 - DNA, 22 bp, Homo sapiens
cctgggccggctctgggatccc

SEQ ID NO: 123 - DNA, 22 bp, Homo sapiens
ctgctggtacctgggccggctc

SEQ ID NO: 124 - DNA, 22 bp, Homo sapiens
tggggatggggcggtcaggcgg

SEQ ID NO: 125 - DNA, 22 bp, Homo sapiens
ccgggggtggggggtggggggt

SEQ ID NO: 126 - DNA, 22 bp, Homo sapiens
cgttttccgggggtggggggtg

SEQ ID NO: 127 - DNA, 22 bp, Homo sapiens
gacgacgcgttttccgggggtg

SEQ ID NO: 128 - DNA, 22 bp, Homo sapiens
cccaggggacgacgcgttttcc

SEQ ID NO: 129 - DNA, 22 bp, Homo sapiens
ggaaaacgcgtcgtcccctggg

SEQ ID NO: 130 - DNA, 22 bp, Homo sapiens
gctgaccgttttcccggagggc

SEQ ID NO: 131 - DNA, 22 bp, Homo sapiens
ctgggccccggaaccggggcga

SEQ ID NO: 132 - DNA, 22 bp, Homo sapiens
ctccctgggccccggaaccggg

SEQ ID NO: 133 - DNA, 22 bp, Homo sapiens
ttcgccccggttccggggccca

SEQ ID NO: 134 - DNA, 22 bp, Homo sapiens
ctccgggacaaaagaccgggac

SEQ ID NO: 135 - DNA, 22 bp, Homo sapiens
aagaccgggactcgggttgccg

SEQ ID NO: 136 - DNA, 22 bp, Homo sapiens
ggatgtgcggtctgtgaaccgc

SEQ ID NO: 137 - DNA, 22 bp, Homo sapiens
gccgcgttgcagggctcagcct

SEQ ID NO: 138 - DNA, 22 bp, Homo sapiens
gggcacccggaaacatgcaggg

SEQ ID NO: 139 - DNA, 22 bp, Homo sapiens
attcccgcgtgcggcaacgtgg

SEQ ID NO: 140 - DNA, 22 bp, Homo sapiens
actcccccacgttgccgcacgc

SEQ ID NO: 141 - DNA, 22 bp, Homo sapiens
tccccggcgtgatggcctgacg

SEQ ID NO: 142 - DNA, 22 bp, Homo sapiens
gagtgtggaactgaacctccgt

SEQ ID NO: 143 - DNA, 22 bp, Homo sapiens
aaaccagcctgggagggtggag

SEQ ID NO: 144 - DNA, 22 bp, Homo sapiens
cagcagggagaaaccagcctgg

SEQ ID NO: 145 - DNA, 22 bp, Homo sapiens
ctcgcagggcctcgctttggct

SEQ ID NO: 146 - DNA, 22 bp, Homo sapiens
tctctggtggcgatgcccgggt

SEQ ID NO: 147 - DNA, 22 bp, Homo sapiens
agccgttctctggtggcgatgc

SEQ ID NO: 148 - DNA, 22 bp, Homo sapiens
accaaatctggaccctgggctc

SEQ ID NO: 149 - DNA, 22 bp, Homo sapiens
gcccagggtccagatttggttt

SEQ ID NO: 150 - DNA, 22 bp, Homo sapiens
cgccagctgaggcagcaccggc

SEQ ID NO: 151 - DNA, 22 bp, Homo sapiens
ggctcggaggagcagggcggtc

SEQ ID NO: 152 - DNA, 22 bp, Homo sapiens
cctgctcctccgagcctttgag

SEQ ID NO: 153 - DNA, 22 bp, Homo sapiens
ctggccagagagacgggcctcc

SEQ ID NO: 154 - DNA, 22 bp, Homo sapiens
cccttcgattctgaaaccagat

SEQ ID NO: 155 - DNA, 22 bp, Homo sapiens
gtccaggattcagatctggttt

SEQ ID NO: 156 - DNA, 22 bp, Homo sapiens
tcgaagggccaggcacccggga

SEQ ID NO: 157 - DNA, 22 bp, Homo sapiens
gcgggcgccctgccaccctgtc

SEQ ID NO: 158 - DNA, 22 bp, Homo sapiens
gcgaaggcgacccacgagggag

SEQ ID NO: 159 - DNA, 22 bp, Homo sapiens
gcgggggtcaccctgctccctc

SEQ ID NO: 160 - DNA, 22 bp, Homo sapiens
agccccaggcgcgcagggcacg

SEQ ID NO: 161 - DNA, 22 bp, Homo sapiens
accgggcctagacctagaaggc

SEQ ID NO: 162 - DNA, 22 bp, Homo sapiens
gcgttttccgggggtggggggt

SEQ ID NO: 163 - DNA, 22 bp, Homo sapiens
cgtccccggtgtgcgccgggcc

SEQ ID NO: 164 - DNA, 22 bp, Homo sapiens
cgggtgaagacccgacggcaac

SEQ ID NO: 165 - DNA, 22 bp, Homo sapiens
ggatgtggggtctgtgaaccgc

SEQ ID NO: 166 - DNA, 22 bp, Homo sapiens
gactccccacgttgccgcacgc

SEQ ID NO: 167 - DNA, 22 bp, Homo sapiens
ggtggtggtggtggtggggggg

SEQ ID NO: 168 - DNA, 22 bp, Homo sapiens
ccagccaggccgcgccggcaga

SEQ ID NO: 169 - DNA, 22 bp, Homo sapiens
accgggcctggacctagaaggc

SEQ ID NO: 170 - DNA, 22 bp, Homo sapiens
tggggagggggcggtcaggcgg

SEQ ID NO: 171 - DNA, 22 bp, Homo sapiens
gattcccgcgtgcggcaacgtg

SEQ ID NO: 172 - DNA, 22 bp, Homo sapiens
tggtggtggtggtggtgggggg

SEQ ID NO: 173 - DNA, 22 bp, Homo sapiens
agaaaggcagttctccgcggag

SEQ ID NO: 174 - DNA, 22 bp, Homo sapiens
aggaaagaaaggcagttctccg

SEQ ID NO: 175 - DNA, 22 bp, Homo sapiens
gcctttctttcctgggcatccc

SEQ ID NO: 176 - DNA, 22 bp, Homo sapiens
gcgagctcccttgcacgtcagc

SEQ ID NO: 177 - DNA, 22 bp, Homo sapiens
ttgttcttccgtgaaattctgg

SEQ ID NO: 178 - DNA, 22 bp, Homo sapiens
aaggtggggggagacattcagc

SEQ ID NO: 179 - DNA, 22 bp, Homo sapiens
ttccgacgctgtctaggcaaac

SEQ ID NO: 180 - DNA, 22 bp, Homo sapiens
cgctgtctaggcaaacctggat

SEQ ID NO: 181 - DNA, 22 bp, Homo sapiens
acctggattagagttacatctc

SEQ ID NO: 182 - DNA, 22 bp, Homo sapiens
tatattaaaatgccccctccct

(19) United States
(12) Patent Application Publication
(10) Pub. No.: US 2023/0323456 A1
(43) Pub. Date: Oct. 12, 2023

(54) METHOD FOR TREATING FACIOSCAPULOHUMERAL MUSCULAR DYSTROPHY (FSHD) BY TARGETING DUX4 GENE

(71) Applicant: MODALIS THERAPEUTICS CORPORATION, Chuo-ku (JP)

(72) Inventors: Tetsuya YAMAGATA, Waltham, MA (US); Yuanbo QIN, Waltham, MA (US); Rebecca WINDMUELLER, Waltham, MA (US)

(73) Assignee: MODALIS THERAPEUTICS CORPORATION, Chuo-ku (JP)

(21) Appl. No.: 18/043,124

(22) PCT Filed: Aug. 31, 2021
(86) PCT No.: PCT/JP2021/031973

§ 371 Date: Feb. 27, 2023

Related U.S. Application Data
(60) Provisional application No. 63/072,327, filed on Aug. 31, 2020.
(60) Provisional application No. 63/235,359, filed on Aug. 20, 2021.

(51) Int. Cl. C12Q 1/6883; C12N 15/11; C12N 15/861

(52) U.S. Cl. CPC C12Q 1/6883; C12N 15/11; C12N 15/861

(57) ABSTRACT

A polynucleotide, comprising the following base sequences: (a) a base sequence encoding a fusion protein of a nuclease-deficient CRISPR effector protein and a transcription repressor, and (b) a base sequence encoding a guide RNA targeting a continuous region set forth in SEQ ID NO: 2, 3, 4, 20, 51, 68, 144, 148, 152, 162, 164, or 167 in the expression regulatory region of human DUX4 gene is expected to be useful for treating or preventing facioscapulohumeral muscular dystrophy (FSHD).

### TECHNICAL FIELD

The present invention relates to methods for treating facioscapulohumeral muscular dystrophy (FSHD) by targeting the human Double homeobox, 4 (DUX4) gene, and the like. More particularly, the present invention relates to methods and agents for treating or preventing FSHD by suppressing expression of human DUX4 gene by using a guide RNA targeting a particular sequence of human DUX4 gene and a fusion protein of a transcription inhibitor and a CRISPR effector protein, and the like.

### BACKGROUND ART

Facioscapulohumeral muscular dystrophy (FSHD) is one of the most prevalent myopathies, affecting males and females of all ages.

There are two types of FSHD. “FSHD1” is attributed to the shortened repeats (10 repeats or less) of the genomic sequence (D4Z4) of telomere (4q35) on chromosome 4, and “FSHD2” is attributed to a complex factor other than FSHD1. DNA is highly methylated in the normal D4Z4 repeat sequence. In FSHD1 and FSHD2, the chromatin structure changes accompanying DNA hypomethylation due to the respective genomic abnormalities, and a gene (DUX4 transcription factor) that is not originally expressed in muscle (progenitor) cells is activated. While DUX4 protein is important in the developmental stage, it is not generally present in mature cells, and DUX4 activation in FSHD skeletal muscle is known to cause cell death. Prevention of the activation of DUX4 is expected to lead to the treatment of FSHD, and as a part thereof, attempts have been made to reduce the amount of DUX4 mRNA by using the gene editing technology (non-patent document 1).

On the other hand, a system using a combination of Cas9 with deactivated nuclease activity (dCas9) and a transcription activation domain or transcription repression domain has been developed in recent years, in which expression of a target gene is controlled through targeting of the protein to the gene by using guide RNA and without cleaving DNA sequence of the gene (patent document 1, which is incorporated herein by reference in its entirety). Its clinical application is expected (see non-patent document 2, which is incorporated herein by reference in its entirety). However, a problem exists in that a sequence encoding a complex of dCas9, guide RNA and a co-transcription repressor exceeds the capacity of the most common viral vectors (e.g., AAV), which represent the most promising method for gene delivery in vivo (see non-patent document 3, which is incorporated herein by reference in its entirety).

### CITATION LIST

### Patent Literature

[PTL 1] WO2013/176772
Non Patent Literature
[NPL 1] Mol Ther. 2016 March; 24(3): 527-535
[NPL 2] Dominguez A. et al., Nat Rev Mol Cell Biol. 2016 January; 17(1): 5-15
[NPL 3] Liao H. et al., Cell. 2017 Dec. 14; 171(7): 1495-507

### SUMMARY OF INVENTION

### Technical Problem

Accordingly, it is one object of the present invention to provide novel therapeutic approaches to Facioscapulohumeral muscular dystrophy (FSHD).

This and other objects, which will become apparent during the following detailed description, have been achieved by the inventors' discovery that the expression of human DUX4 gene (Gene ID: 100288687) can be strongly suppressed by using a guide RNA targeting a particular sequence of human DUX4 gene and a fusion protein of a transcription repressor and a nuclease-deficient CRISPR effector protein. In addition, the present inventors have found that the expression of human DUX4 gene can be strongly suppressed by a single AAV vector carrying a base sequence encoding the fusion protein and a base sequence encoding the guide RNA, using a compact nuclease-deficient CRISPR effector protein and a compact transcription repressor.

Thus, the present invention provides:

[1] A polynucleotide, comprising the following base sequences:
(a) a base sequence encoding a fusion protein of a nuclease-deficient CRISPR effector protein and a transcription repressor, and
(b) a base sequence encoding a guide RNA targeting a continuous region set forth in SEQ ID NO: 2, 3, 4, 20, 51, 68, 138, 142, 146, 156, 158, or 161 in the expression regulatory region of human DUX4 gene.

[2] The polynucleotide of [1], wherein the base sequence encoding the guide RNA comprises the base sequence set forth in SEQ ID NO: 2, 3, 4, 20, 51, 68, 138, 142, 146, 156, 158, or 161, or the base sequence set forth in SEQ ID NO: 2, 3, 4, 20, 51, 68, 138, 142, 146, 156, 158, or 161 in which 1 to 3 bases are deleted, substituted, inserted, and/or added.

[3] The polynucleotide of [1] or [2], comprising at least two different base sequences encoding the guide RNA.

[4] The polynucleotide of any of [1] to [3], wherein the transcriptional repressor is selected from the group KRAB, MeCP2, SIN3A, HDT1, MBD2B, NIPP1, and HP1A.

[5] The polynucleotide of [4], wherein the transcriptional repressor is KRAB.

[6] The polynucleotide of any of [1] to [5], wherein the nuclease-deficient CRISPR effector protein is dCas9.

[7] The polynucleotide of [6], wherein the dCas9 is derived from Staphylococcus aureus.

[8] The polynucleotide of any of [1] to [7], further comprising a promoter sequence for the base sequence encoding the guide RNA and/or a promoter sequence for the base sequence encoding the fusion protein of the nuclease-deficient CRISPR effector protein and the transcriptional repressor.

[9] The polynucleotide of [8], wherein the promoter sequence for the base sequence encoding the guide RNA is selected from the group U6 promoter, SNR6 promoter, SNR52 promoter, SCR1 promoter, RPR1 promoter, U3 promoter, and H1 promoter.

[10] The polynucleotide of [9], wherein the promoter sequence for the base sequence encoding the guide RNA is U6 promoter.

[11] The polynucleotide of any of [8] to [10], wherein the promoter sequence for the base sequence encoding the fusion protein of the nuclease-deficient CRISPR effector protein and the transcriptional repressor is a ubiquitous promoter or a neuron specific promoter.

[12] The polynucleotide of [11], wherein the ubiquitous promoter is selected from the group EFS promoter, CMV promoter and CAG promoter.

[13] A vector comprising a polynucleotide of any of [1] to [12].

[14] The vector of [13], wherein the vector is a plasmid vector or a viral vector.

[15] The vector of [14], wherein the viral vector is selected from the group adeno-associated virus (AAV) vector, adenovirus vector, and lentivirus vector.

[16] The vector of [15], wherein the AAV vector is selected from the group AAV1, AAV2, AAV6, AAV7, AAV8, AAV9, Anc80, AAV₅₈₇MTP, AAV₅₈₈MTP, AAV-B1, AAVM41, and AAVrh74.

[17] The vector of [16], wherein the AAV vector is AAV9.

[18] A pharmaceutical composition comprising a polynucleotide of any of [1] to [12] or a vector of any of [13] to [17].

[19] The pharmaceutical composition of [18] for treating or preventing FSHD.

[20] A method for treating or preventing FSHD, comprising administering a polynucleotide of any of [1] to [12], or a vector of any of [13] to [17], to a subject in need thereof.

A more complete appreciation of the invention and many of the attendant advantages thereof will be readily obtained as the same become better understood by reference to the following detailed description when considered in connection with the accompanying drawings.

### Advantageous Effects of Invention

According to the present invention, the expression of the human DUX4 gene can be suppressed and, consequently, the present invention is expected to be able to treat FSHD.

### BRIEF DESCRIPTION OF DRAWINGS

FIG. 1 shows the location of the targeted genome regions relative to human DUX4 gene.

FIG. 2 shows the evaluation results of an expression suppressing action on human DUX4 gene in two lymphoblast cell lines (LCLs; GM16343 LCL and GM16414 LCL) derived from FSHD patients by using sgRNA containing crRNA encoded by the targeting sequence shown in SEQ ID NOs: 1 to 76. The horizontal axis shows sgRNA containing crRNA encoded by each targeting sequence, and the vertical axis shows the ratio of the expression level of DUX4 gene when using each sgRNA to that when using control sgRNA as 1.

FIG. 3A shows the evaluation results of an expression suppressing action on human DUX4 gene in GM16343 LCL by using sgRNA containing crRNA encoded by the selected 27 targeting sequences. The horizontal axis shows sgRNA containing crRNA encoded by each targeting sequence, and the vertical axis shows the ratio of the expression level of DUX4 gene when using each sgRNA to that when using control sgRNA as 1.

FIG. 3B shows the evaluation results of an expression suppressing action on human DUX4 gene in GM16414 LCL by using sgRNA containing crRNA encoded by the selected 27 targeting sequences. The horizontal axis shows sgRNA containing crRNA encoded by each targeting sequence, and the vertical axis shows the ratio of the expression level of DUX4 gene when using each sgRNA to that when using control sgRNA as 1.

FIG. 4 shows the evaluation results of quantification of DUX4 and FSHD biomarkers TRIM43, MBD3L2 and ZSCAN4 from the best 6 sgRNAs identified in validation experiment using FSHD patient derived LCLs. The horizontal axis shows sgRNA containing crRNA encoded by each targeting sequence, and the vertical axis shows the ratio of the expression level of DUX4 gene when using each sgRNA to that when using control sgRNA as 1.

FIG. 5 shows the location of the targeted genome regions relative to human DUX4 gene.

FIG. 6 shows the evaluation results of an expression suppressing action on human DUX4 gene in one lymphoblast cell line (LCLs; GM16343 LCL) derived from FSHD patients by using sgRNA containing crRNA encoded by the targeting sequence shown in SEQ ID NOs: 104 to 188. The horizontal axis shows sgRNA containing crRNA encoded by each targeting sequence, and the vertical axis shows the ratio of the expression level of DUX4 gene when using each sgRNA to that when using control sgRNA as 1.

FIG. 7 shows the evaluation results of quantification of DUX4 and FSHD biomarkers TRIM43, MBD3L2 and ZSCAN4 from the best 6 sgRNAs identified in validation experiment using FSHD patient derived LCLs (N=2). The horizontal axis shows sgRNA containing crRNA encoded by each targeting sequence, and the vertical axis shows the ratio of the expression level of DUX4 gene when using each sgRNA to that when using control sgRNA as 1.

### DESCRIPTION OF EMBODIMENTS

The embodiments of the present invention are explained in detail below.

1. Polynucleotide

The present invention provides a polynucleotide comprising the following base sequences (hereinafter sometimes to be also referred to as “the polynucleotide of the present invention”):
(a) a base sequence encoding a fusion protein of a nuclease-deficient CRISPR effector protein and a transcription repressor, and
(b) a base sequence encoding a guide RNA targeting a continuous region set forth in SEQ ID NO: 2, 3, 4, 20, 51, 68, 138, 142, 146, 156, 158, or 161 in the expression regulatory region of human DUX4 gene.

The polynucleotide of the present invention is introduced into a desired cell and transcribed to produce a fusion protein of a nuclease-deficient CRISPR effector protein and a transcription repressor, and a guide RNA targeting a particular region of the expression regulatory region of the human DUX4 gene. These fusion protein and guide RNA form a complex (hereinafter the complex is sometimes referred to as “ribonucleoprotein; RNP”) and cooperatively act on the aforementioned particular region, thus suppressing transcription of the human DUX4 gene. In one embodiment of the present invention, the expression of the human DUX4 gene can be suppressed by, for example, not less than about 40%, not less than about 50%, not less than about 60%, not less than about 70%, not less than about 75%, not less than about 80%, not less than about 85%, not less than about 90%, not less than about 95%, or about 100%.

### (1) Definition

In the present specification, “the expression regulatory region of human Double homeobox, 4 (DUX4) gene” means any region in which the expression of human DUX4 gene can be suppressed by binding RNP to that region. That is, the expression regulatory region of human DUX4 gene may exist in any region such as the promoter region, enhancer region, intron, and exon of the human DUX4 gene, as long as the expression of the human DUX4 gene is suppressed by the binding of RNP. In the present specification, when the expression regulatory region is shown by the particular sequence, the expression regulatory region includes both the sense strand sequence and the antisense strand sequence conceptually.

In the present invention, a fusion protein of a nuclease-deficient CRISPR effector protein and a transcription repressor is recruited by a guide RNA into a particular region in the expression regulatory region of the human DUX4 gene. In the present specification, the “guide RNA targeting . . . ” means a “guide RNA recruiting a fusion protein into . . . ”.

In the present specification, the “guide RNA (to be also referred to as ‘gRNA’)” is an RNA comprising a genome specific CRISPR-RNA (to be referred to as “crRNA”). crRNA is an RNA that binds to a complementary sequence of a targeting sequence (described later). When Cpf1 is used as the CRISPR effector protein, the “guide RNA” refers to an RNA comprising an RNA consisting of crRNA and a specific sequence attached to its 5′-terminal (for example, an RNA sequence set forth in SEQ ID NO: 80 in the case of FnCpf 1). When Cas9 is used as the CRISPR effector protein, the “guide RNA” refers to chimera RNA (to be referred to as “single guide RNA (sgRNA)”) comprising crRNA and trans-activating crRNA attached to its 3′-terminal (to be referred to as “tracrRNA”) (see, for example, Zhang F. et al., Hum Mol Genet. 2014 Sep. 15; 23(R1): R40-6 and Zetsche B. et al., Cell. 2015 Oct. 22; 163(3): 759-71, which are incorporated herein by reference in their entireties).

In the present specification, a sequence complementary to the sequence to which crRNA is bound in the expression regulatory region of the human DUX4 gene is referred to as a “targeting sequence”. That is, in the present specification, the “targeting sequence” is a DNA sequence present in the expression regulatory region of the human DUX4 gene and adjacent to PAM (protospacer adjacent motif). PAM is adjacent to the 5′-side of the targeting sequence when Cpf1 is used as the CRISPR effector protein. PAM is adjacent to the 3′-side of the targeting sequence when Cas9 is used as the CRISPR effector protein. The targeting sequence may be present on either the sense strand sequence side or the antisense strand sequence side of the expression regulatory region of the human DUX4 gene (see, for example, the aforementioned Zhang F. et al., Hum Mol Genet. 2014 Sep. 15; 23(R1): R40-6 and Zetsche B. et al., Cell. 2015 Oct. 22; 163(3): 759-71, which are incorporated herein by reference in their entireties).

### (2) Nuclease-Deficient CRISPR Effector Protein

In the present invention, using a nuclease-deficient CRISPR effector protein, a transcriptional repressor fused thereto is recruited to the expression regulatory region of the human DUX4 gene. The nuclease-deficient CRISPR effector protein (hereinafter to be simply referred to as “CRISPR effector protein”) to be used in the present invention is not particularly limited as long as it forms a complex with gRNA and is recruited to the expression regulatory region of the human DUX4 gene. For example, nuclease-deficient Cas9 (hereinafter sometimes to be also referred to as “dCas9”) or nuclease-deficient Cpf1 (hereinafter sometimes to be also referred to as “dCpf1”) can be included.

Examples of the above-mentioned dCas9 include, but are not limited to, a nuclease-deficient variant of Streptococcus pyogenes-derived Cas9 (SpCas9; PAM sequence: NGG (N is A, G, T or C. hereinafter the same)), Streptococcus thermophilus-derived Cas9 (StCas9; PAM sequence: NNAGAAW (W is A or T. hereinafter the same)), Neisseria meningitidis-derived Cas9 (NmCas9; PAM sequence: NNNNGATT), or Staphylococcus aureus-derived Cas9 (SaCas9; PAM sequence: NNGRRT (R is A or G. hereinafter the same)) and the like (see, for example, Nishimasu et al., Cell. 2014 Feb. 27; 156(5): 935-49, Esvelt K M et al., Nat Methods. 2013 November; 10(11):1116-21, Zhang Y. Mol Cell. 2015 Oct. 15; 60(2):242-55, and Friedland A E et al., Genome Biol. 2015 Nov. 24; 16:257, which are incorporated herein by reference in their entireties). For example, in the case of SpCas9, a double mutant in which the 10th Asp residue is converted to Ala residue and the 840th His residue is converted to Ala residue (sometimes referred to as “dSpCas9”) can be used (see, for example, the aforementioned Nishimasu et al., Cell. 2014). Alternatively, in the case of SaCas9, a double mutant in which the 10th Asp residue is converted to Ala residue and the 580th Asn residue is converted to Ala residue (SEQ ID NO: 81), or a double mutant in which the 10th Asp residue is converted to Ala residue and the 557th His residue is converted to Ala residue (SEQ ID NO: 82) (hereinafter any of these double mutants is sometimes to be referred to as “dSaCas9”) can be used (see, for example, the aforementioned Friedland A E et al., Genome Biol. 2015, which is incorporated herein by reference in its entirety).

In addition, in one embodiment of the present invention, as dCas9, a variant obtained by modifying a part of the amino acid sequence of the aforementioned dCas9, which forms a complex with gRNA and is recruited to the expression regulatory region of the human DUX4 gene, may also be used. Examples of such variants include a truncated variant with a partly deleted amino acid sequence. In one embodiment of the present invention, as dCas9, variants disclosed in WO2019/235627 and WO2020/085441, which are incorporated herein by reference in their entireties, can be used. Specifically, dSaCas9 obtained by deleting the 721st to 745th amino acids from dSaCas9 that is a double mutant in which the 10th Asp residue is converted to Ala residue and the 580th Asn residue is converted to Ala residue (SEQ ID NO: 83), or dSaCas9 in which the deleted part is substituted by a peptide linker (e.g., one in which the deleted part is substituted by GGSGGS linker (SEQ ID NO: 84) is set forth in SEQ ID NO: 85, and one in which the deleted part is substituted by SGGGS linker (SEQ ID NO: 86) is set forth in SEQ ID NO: 87, etc.) (hereinafter any of these double mutants is sometimes to be referred to as “dSaCas9[−25]”), or dSaCas9 obtained by deleting the 482nd to 648th amino acids from dSaCas9 that is the aforementioned double mutant (SEQ ID NO: 88), or dSaCas9 in which the deleted part is substituted by a peptide linker (one in which the deleted part is substituted by GGSGGS linker is set forth in SEQ ID NO: 89) may also be used.

Examples of the above-mentioned dCpf1 include, but are not limited to, a nuclease-deficient variant of Francisella novicida-derived Cpf1 (FnCpf1; PAM sequence: NTT), Acidaminococcus sp.-derived Cpf1 (AsCpf1; PAM sequence: NTTT), or Lachnospiraceae bacterium-derived Cpf1 (LbCpf1; PAM sequence: NTTT) and the like (see, for example, Zetsche B. et al., Cell. 2015 Oct. 22; 163(3):759-71, Yamano T et al., Cell. 2016 May 5; 165(4):949-62, and Yamano T et al., Mol Cell. 2017 Aug. 17; 67(4):633-45, which are incorporated herein by reference in their entireties). For example, in the case of FnCpf1, a double mutant in which the 917th Asp residue is converted to Ala residue and the 1006th Glu residue is converted to Ala residue can be used (see, for example, the aforementioned Zetsche B et al., Cell. 2015, which is incorporated herein by reference in its entirety). In one embodiment of the present invention, as dCpf1, a variant obtained by modifying a part of the amino acid sequence of the aforementioned dCpf1, which forms a complex with gRNA and is recruited to the expression regulatory region of the human DUX4 gene, may also be used.

In one embodiment of the present invention, dCas9 is used as the nuclease-deficient CRISPR effector protein. In one embodiment, the dCas9 is dSaCas9, and, in a particular embodiment, the dSaCas9 is dSaCas9[−25].

A polynucleotide comprising a base sequence encoding a CRISPR effector protein can be cloned by, for example, synthesizing an oligoDNA primer covering a region encoding a desired part of the protein based on the cDNA sequence information thereof, and amplifying the polynucleotide by PCR method using total RNA or mRNA fraction prepared from the cells producing the protein as a template. In addition, a polynucleotide comprising a base sequence encoding a nuclease-deficient CRISPR effector protein can be obtained by introducing a mutation into a nucleotide sequence encoding a cloned CRISPR effector protein by a known site-directed mutagenesis method to convert the amino acid residues (e.g., 10th Asp residue, 557th His residue, and 580th Asn residue in the case of SaCas9; 917th Asp residue and 1006th Glu residue in the case of FnCpf1, and the like can be included, but are not limited to these) at a site important for DNA cleavage activity to other amino acids.

Alternatively, a polynucleotide comprising a base sequence encoding nuclease-deficient CRISPR effector protein can be obtained by chemical synthesis or a combination of chemical synthesis and PCR method or Gibson Assembly method, based on the cDNA sequence information thereof, and can also be further constructed as a base sequence that underwent codon optimization to give codons suitable for expression in human.

### (3) Transcriptional Repressor

In the present invention, human DUX4 gene expression is repressed by the action of the transcriptional repressor fused with the nuclease-deficient CRISPR effector protein. In the present specification, the “transcriptional repressor” means a protein having the ability to repress gene transcription of human DUX4 gene or a peptide fragment retaining the function thereof. The transcriptional repressor to be used in the present invention is not particularly limited as long as it can repress expression of human DUX4 gene. It includes, for example, Kruppel-associated box (KRAB), MBD2B, v-ErbA, SID (including chain state of SID (SID4X)), MBD2, MBD3, DNMT family (e.g., DNMT1, DNMT3A, DNMT3B), Rb, MeCP2, ROM2, LSD1, AtHD2A, SET1, HDAC11, SETD8, EZH2, SUV39H1, PHF19, SALI, NUE, SUVR4, KYP, DIM5, HDAC8, SIRT3, SIRT6, MESOLO4, SET8, HST2, COBB, SET-TAF1B, NCOR, SIN3A, HDT1, NIPP1, HP1A, ERF repressor domain (ERD), and variants thereof having transcriptional repression ability, fusions thereof and the like. In one embodiment of the present invention, KRAB is used as the transcriptional repressor.

A polynucleotide comprising a base sequence encoding a transcriptional repressor can be constructed by chemical synthesis or a combination of chemical synthesis and PCR method or Gibson Assembly method. Furthermore, a polynucleotide comprising a base sequence encoding a transcriptional repressor can also be constructed as a codon-optimized DNA sequence to be codons suitable for expression in human.

A polynucleotide comprising a base sequence encoding a fusion protein of a transcriptional repressor and a nuclease-deficient CRISPR effector protein can be prepared by ligating a base sequence encoding the CRISPR effector protein to a base sequence encoding the transcriptional repressor directly or after adding a base sequence encoding a linker, NLS (nuclear localization signal)(for example, a base sequence set forth in SEQ ID NO: 90 or SEQ ID NO: 91), a tag and/or others. In the present invention, the transcriptional repressor may be fused with either N-terminal or C-terminal of the nuclease-deficient CRISPR effector protein. As the linker, a linker with an amino acid number of about 2 to 50 can be used, and specific examples thereof include, but are not limited to, a G-S-G-S linker in which glycine (G) and serine (S) are alternately linked and the like. In one embodiment of the present invention, as the polynucleotide comprising a base sequence encoding a fusion protein of a nuclease-deficient CRISPR effector protein and a transcriptional repressor, the base sequence set forth in SEQ ID NO: 92, which encodes SV40 NLS, dSaCas9 (e.g., D10A and N580A mutant), NLS and KRAB as a fused protein, can be used. If desired, other base sequence (see “(6) other base sequences” below) and selection marker (e.g., Puro) may be contained.

### (4) Guide RNA

In the present invention, a fusion protein of nuclease-deficient CRISPR effector protein and transcription repressor can be recruited to the expression regulatory region of the human DUX4 gene by guide RNA. As described in the aforementioned “(1) Definition”, guide RNA comprises crRNA, and the crRNA binds to a complementary sequence of the targeting sequence. crRNA may not be completely complementary to the complementary sequence of the targeting sequence as long as the guide RNA can recruit the fusion protein to the target region, and may comprise a base sequence of the targeting sequence in which at least 1 to 3 bases are deleted, substituted, inserted and/or added.

When dCas9 is used as the nuclease-deficient CRISPR effector protein, for example, the targeting sequence can be determined using a published gRNA design web site (CRISPR Design Tool, CRISPR direct, etc.). To be specific, from the sequence of the target gene (i.e., human DUX4 gene), candidate targeting sequences of about 20 nucleotides in length for which PAM (e.g., NNGRRT in the case of SaCas9) is adjacent to the 3′-side thereof are listed, and one having a small number of off-target sites in human genome from among these candidate targeting sequences can be used as the targeting sequence. The base length of the targeting sequence is 18 to 24 nucleotides in length, preferably 20 to 23 nucleotides in length, more preferably 21 to 23 nucleotides in length. As a primary screening for the prediction of the off-target site number, a number of bioinformatic tools are known and publicly available, and can be used to predict the targeting sequence with the lowest off-target effect. Examples thereof include bioinformatics tools such as Benchling (https://benchling.com), and COSMID (CRISPR Off-target Sites with Mismatches, Insertions, and Deletions) (Available on https://crispr.bme.gatech.edu on the internet). Using these, the similarity to the base sequence targeted by gRNA can be summarized. When the gRNA design software to be used does not have a function to search for off-target site of the target genome, for example, the off-target site can be searched for by subjecting the target genome to Blast search with respect to 8 to 12 nucleotides on the 3′-side of the candidate targeting sequence (seed sequence with high discrimination ability of targeted nucleotide sequence).

In one embodiment of the present invention, in the region existing in the GRCh38/hg38 of human chromosome 4 (Chr 4), the region of “190,065,500-190,068,500” and the region of “190,047,000-190,052,000” can be the expression regulatory region of the human DUX4 gene. Therefore, in one embodiment of the present invention, the targeting sequence can be 18 to 24 nucleotides in length, preferably 20 to 23 nucleotides in length, more preferably 21 to 23 nucleotides in length, in the regions of “190,065,500-190,068,500” and “190,047,000-190,052,000” existing in the GRCh38/hg38 of human chromosome 4 (Chr 4).

In one embodiment of the present invention, the targeting sequence can be 18 to 24 nucleotides in length, preferably 20 to 23 nucleotides in length, more preferably 21 to 23 nucleotides in length, in the regions of “190,065,000-190,093,000” (D4Z4 repeat region) and “190,173,000-190,176,000” (DUX4 gene) existing in the GRCh38/hg38 of human chromosome 4 (Chr 4).

In one embodiment of the present invention, a base sequence encoding crRNA may be the same base sequence as the targeting sequence. For example, when the targeting sequence set forth in SEQ ID NO: 4 (CCCTCCACCGGGCTGACCGGCC) is introduced into the cell as a base sequence encoding crRNA, crRNA transcribed from the sequence is CCCUCCACCGGGCUGACCGGCC (SEQ ID NO: 93) and is bound to GGCCGGTCAGCCCGGTGGAGGG (SEQ ID NO: 94), which is a sequence complementary to the base sequence set forth in SEQ ID NO: 4 and is present in the expression regulatory region of the human DUX4 gene. In another embodiment, a base sequence which is a targeting sequence in which at least 1 to 3 bases are deleted, substituted, inserted and/or added can be used as the base sequence encoding crRNA as long as guide RNA can recruit a fusion protein to the target region. Therefore, in one embodiment of the present invention, as a base sequence encoding crRNA, the base sequence set forth in SEQ ID NO: 2, 3, 4, 8, 15, 17, 18, 20, 25, 31, 32, 33, 35, 39, 40, 42, 44, 50, 51, 52, 55, 57, 58, 59, 65, 67, 68, 113, 116, 135, 138, 142, 144, 146, 156, 158, 161, or 171 or the base sequence set forth in SEQ ID NO: 2, 3, 4, 8, 15, 17, 18, 20, 25, 31, 32, 33, 35, 39, 40, 42, 44, 50, 51, 52, 55, 57, 58, 59, 65, 67, 68, 113, 116, 135, 138, 142, 144, 146, 156, 158, 161, or 171 in which 1 to 3 bases are deleted, substituted, inserted and/or added can be used.

In one preferable embodiment of the present invention, as a base sequence encoding crRNA, the base sequence set forth in SEQ ID NO: 2, 3, 4, 20, 51, 68, 138, 142, 146, 156, 158, or 161, or the base sequence set forth in SEQ ID NO: 2, 3, 4, 20, 51, 68, 138, 142, 146, 156, 158, or 161 in which 1 to 3 bases are deleted, substituted, inserted and/or added can be used.

When dCpf1 is used as the nuclease-deficient CRISPR effector protein, a base sequence encoding gRNA can be designed as a DNA sequence encoding crRNA with particular RNA attached to the 5′-terminal. Such RNA attached to the 5′-terminal of crRNA and a DNA sequence encoding said RNA can be appropriately selected by those of ordinary skill in the art according to the dCpf1 to be used. For example, when dFnCpf1 is used, a base sequence in which SEQ ID NO: 95; AATTTCTACTGTT GTAGAT is attached to the 5′-side of the targeting sequence can be used as a base sequence encoding gRNA (when transcribed to RNA, the sequences of the underlined parts form base pairs to form a stem-loop structure). The sequence to be added to the 5′-terminal may be a sequence generally used for various Cpf1 proteins in which at least 1 to 6 bases are deleted, substituted, inserted and/or added, as long as gRNA can recruit a fusion protein to the expression regulatory region after transcription.

When dCas9 is used as the CRISPR effector protein, a base sequence encoding gRNA can be designed as a DNA sequence in which a DNA sequence encoding known tracrRNA is linked to the 3′-terminal of a DNA sequence encoding crRNA. Such tracrRNA and a DNA sequence encoding the tracrRNA can be appropriately selected by those of ordinary skill in the art according to the dCas9 to be used. For example, when dSaCas9 is used, the base sequence set forth in SEQ ID NO: 96 is used as the DNA sequence encoding tracrRNA. The DNA sequence encoding tracrRNA may be a base sequence encoding tracrRNA generally used for various Cas9 proteins in which at least 1 to 6 bases are deleted, substituted, inserted and/or added, as long as gRNA can recruit a fusion protein to the expression regulatory region after transcription.

A polynucleotide comprising a base sequence encoding gRNA designed in this way can be chemically synthesized using a known DNA synthesis method.

In another embodiment of the present invention, the polynucleotide of the present invention may comprise at least two different base sequences encoding a gRNA. For example, the polynucleotide can comprise at least two different base sequences encoding the guide RNA, wherein the at least two different base sequences are selected from a base sequence comprising a sequence set forth in SEQ ID NO: 2, 3, 4, 8, 15, 17, 18, 20, 25, 31, 32, 33, 35, 39, 40, 42, 44, 50, 51, 52, 55, 57, 58, 59, 65, 67, 68, 113, 116, 135, 138, 142, 144, 146, 156, 158, 161, or 171, preferably are selected from a base sequence comprising a sequence set forth in SEQ ID NO: 2, 3, 4, 20, 51, 68, 138, 142, 146, 156, 158, or 161.

### (5) Promoter Sequence

In one embodiment of the present invention, a promoter sequence may be operably linked to the upstream of each of a base sequence encoding fusion protein of nuclease-deficient CRISPR effector protein and transcriptional repressor and/or a base sequence encoding gRNA. The promoter to be possibly linked is not particularly limited as long as it shows a promoter activity in the target cell. Examples of the promoter sequence possibly linked to the upstream of the base sequence encoding gRNA include, but are not limited to, U6 promoter, SNR6 promoter, SNR52 promoter, SCR1 promoter, RPR1 promoter, U3 promoter, H1 promoter, and tRNA promoter, which are pol III promoters, and the like. In one embodiment of the present invention, U6 promoter can be used as the promoter sequence for the base sequence encoding the guide RNA. In one embodiment of the present invention, when a polynucleotide comprises two or more base sequences respectively encoding a guide RNA, a single promoter sequence may be operably linked to the upstream of the two or more base sequences. In another embodiment, when a polynucleotide comprises two or more base sequences respectively encoding a guide RNA, a promoter sequence may be operably linked to the upstream of each of the two or more base sequences, wherein the promoter sequence operably linked to each base sequence may be the same or different.

As the aforementioned promoter sequence possibly linked to the upstream of the base sequence encoding fusion protein, a ubiquitous promoter or neuron-specific promoter may be used. Examples of the ubiquitous promoter include, but are not limited to, EF1α promoter, EFS promoter, CMV (cytomegalovirus) promoter, hTERT promoter, SRα promoter, SV40 promoter, LTR promoter, CAG promoter, RSV (Rous sarcoma virus) promoter, and the like. In one embodiment of the present invention, EFS promoter, CMV promoter or CAG promoter can be used as the ubiquitous promoter. Examples of the neuron-specific promoter include, but are not limited to, neuron-specific enolase (NSE) promoter, human neurofilament light chain (NEFL) promoter. The aforementioned promoter may have any modification and/or alteration as long as it has promoter activity in the target cell.

In one embodiment of the present invention, U6 is used as a promoter for a base sequence encoding the guide RNA, and CMV promoter can be used as the promoter sequence for the base sequence encoding the fusion protein.

### (6) Other Base Sequence

Furthermore, the polynucleotide of the present invention may further comprise known sequences such as Polyadenylation (polyA) signal, Kozak consensus sequence and the like besides those mentioned above for the purpose of improving the translation efficiency of mRNA produced by transcription of a base sequence encoding a fusion protein of nuclease-deficient CRISPR effector protein and transcription repressor. For example, Polyadenylation signal in the present invention may include hGH polyA, bGH polyA, 2×sNRP-1 polyA (see U.S. Pat. No. 7,557,197B2, which is incorporated herein by reference in its entirety), and so on. In addition, the polynucleotide of the present invention may comprise a base sequence encoding a linker sequence, a base sequence encoding NLS and/or a base sequence encoding a tag. Futhermore, the polynucleotide of the present invention may comprise an intervening sequence. A preferred example of the intervening sequence is a sequence encoding IRES (Internal ribosome entry site), 2A peptide. The 2A peptide is a peptide sequence of around 20 amino acid residues derived from virus, is recognized by a protease present in the cell (2A peptidase), and is cleaved at the position of 1 residue from the C terminal. Multiple genes linked as one unit by 2A peptide are transcribed and translated as one unit, and then cleaved by 2A peptidase. Examples of the 2A peptidase include F2A (derived from foot-and-mouth disease virus), E2A (derived from equine rhinitis A virus), T2A (derived from Thosea asigna virus), and P2A (derived from porcine teschovirus-1).

### (7) Exemplified Embodiments of the Present Invention

In one embodiment of the present invention, a polynucleotide is provided comprising:
a base sequence encoding a fusion protein of a nuclease-deficient CRISPR effector protein and a transcriptional repressor,
a promoter sequence for the base sequence encoding the fusion protein of the nuclease-deficient CRISPR effector protein and the transcriptional repressor,
one or two base sequences respectively encoding a guide RNA, wherein the one or two base sequences are selected from a base sequence comprising a sequence set forth in SEQ ID NO: 2, 3, 4, 8, 15, 17, 18, 20, 25, 31, 32, 33, 35, 39, 40, 42, 44, 50, 51, 52, 55, 57, 58, 59, 65, 67, 68, 113, 116, 135, 138, 142, 144, 146, 156, 158, 161, or 171, preferably are selected from a base sequence comprising a sequence set forth in SEQ ID NO: 2, 3, 4, 20, 51, 68, 138, 142, 146, 156, 158, or 161; or the base sequence comprising a sequence set forth in SEQ ID NO: 2, 3, 4, 8, 15, 17, 18, 20, 25, 31, 32, 33, 35, 39, 40, 42, 44, 50, 51, 52, 55, 57, 58, 59, 65, 67, 68, 113, 116, 135, 138, 142, 144, 146, 156, 158, 161, or 171, preferably are selected from a base sequence comprising a sequence set forth in SEQ ID NO: 2, 3, 4, 20, 51, 68, 138, 142, 146, 156, 158, or 161 in which 1 to 3 bases are deleted, substituted, inserted, and/or added, and
a promoter sequence for the base sequence encoding the gRNA,
wherein the nuclease-deficient CRISPR effector protein is dSaCas9 or dSaCas9[−25],
wherein the transcriptional repressor is selected from the group KRAB, MeCP2, SIN3A, HDT1, MBD2B, NIPP1, and HP1A,
wherein the promoter sequence for the base sequence encoding the fusion protein is selected from the group EFS promoter, CMV promoter and CAG promoter, and
wherein the promoter sequence for the base sequence encoding the gRNA is selected from the group U6 promoter, SNR6 promoter, SNR52 promoter, SCR1 promoter, RPR1 promoter, U3 promoter, and H1 promoter.

In one embodiment of the present invention, a polynucleotide is provided comprising:
a base sequence encoding a fusion protein of a nuclease-deficient CRISPR effector protein and a transcriptional repressor,
CMV promoter for the base sequence encoding the fusion protein of the nuclease-deficient CRISPR effector protein and the transcriptional repressor,
one or two base sequences respectively encoding a guide RNA, wherein the one or two base sequences are selected from a base sequence comprising a sequence set forth in SEQ ID NO: 2, 3, 4, 8, 15, 17, 18, 20, 25, 31, 32, 33, 35, 39, 40, 42, 44, 50, 51, 52, 55, 57, 58, 59, 65, 67, 68, 113, 116, 135, 138, 142, 144, 146, 156, 158, 161, or 171, preferably are selected from a base sequence comprising a sequence set forth in SEQ ID NO: 2, 3, 4, 20, 51, 68, 138, 142, 146, 156, 158, or 161; or abase sequence comprising a sequence set forth in SEQ ID NO: 2, 3, 4, 8, 15, 17, 18, 20, 25, 31, 32, 33, 35, 39, 40, 42, 44, 50, 51, 52, 55, 57, 58, 59, 65, 67, 68, 113, 116, 135, 138, 142, 144, 146, 156, 158, 161, or 171, preferably are selected from a base sequence comprising a sequence set forth in SEQ ID NO: 2, 3, 4, 20, 51, 68, 138, 142, 146, 156, 158, or 161 in which 1 to 3 bases are deleted, substituted, inserted, and/or added, and U6 promoter for the base sequence encoding the guide RNA,
wherein the nuclease-deficient CRISPR effector protein is dSaCas9, and wherein the transcriptional repressor is KRAB.

2. Vector

The present invention provides a vector comprising the polynucleotide of the present invention (hereinafter sometimes referred to as “the vector of the present invention”). The vector of the present invention may be a plasmid vector or a viral vector.

When the vector of the present invention is a plasmid vector, the plasmid vector to be used is not particularly limited and may be any plasmid vector such as cloning plasmid vector and expression plasmid vector. The plasmid vector is prepared by inserting the polynucleotide of the present invention into a plasmid vector by a known method.

When the vector of the present invention is a viral vector, the viral vector to be used is not particularly limited and examples thereof include, but are not limited to, adenovirus vector, adeno-associated virus (AAV) vector, lentivirus vector, retrovirus vector, Sendaivirus vector and the like. In the present specification, the “virus vector” or “viral vector” also includes derivatives thereof. Considering the use in gene therapy, AAV vector is preferably used for the reasons such that it can express transgene for a long time, and it is derived from a non-pathogenic virus and has high safety.

A viral vector comprising the polynucleotide of the present invention can be prepared by a known method. In brief, a plasmid vector for virus expression into which the polynucleotide of the present invention has been inserted is prepared, the vector is transfected into an appropriate host cell to allow for transient production of a viral vector comprising the polynucleotide of the present invention, and the viral vector is collected.

In one embodiment of the present invention, when AAV vector is used, the serotype of the AAV vector is not particularly limited as long as expression of the human DUX4 gene in the target can be activated, and any of AAV1, AAV2, AAV3, AAV4, AAV5, AAV6, AAV7, AAV8, AAV9, AAVrh.10 and the like may be used (for the various serotypes of AAV, see, for example, WO 2005/033321 and EP2341068 (A1), which are incorporated herein by reference in their entireties). Examples of the variants of AAV include, but are not limited to, new serotype with a modified capsid (e.g., WO 2012/057363, which is incorporated herein by reference in its entirety) and the like. For example, in one embodiment of the present invention, a new serotype with a modified capsid improving infectivity for muscle cells can be used, such as AAV₅₈₇ MTP, AAV₅₈₈MTP, AAV-B1, AAVM41, AAVS1_P1, and AAVS10_P1, and the like (see Yu et al., Gene Ther. 2009 August; 16(8):953-62, Choudhury et al., Mol Ther. 2016 August; 24(7):1247-57, Yang et al., Proc Natl Acad Sci USA. 2009 Mar. 10; 106(10):3946-51, and WO2019/207132, which are incorporated herein by reference in their entireties).

When an AAV vector is prepared, a known method such as (1) a method using a plasmid, (2) a method using a baculovirus, (3) a method using a herpes simplex virus, (4) a method using an adenovirus, or (5) a method using yeast can be used (e.g., Appl Microbiol Biotechnol. 2018; 102(3): 1045-1054, etc., which is incorporated herein by reference in its entirety). For example, when an AAV vector is prepared by a method using a plasmid, first, a vector plasmid comprising inverted terminal repeat (ITR) at both ends of wild-type AAV genomic sequence and the polynucleotide of the present invention inserted in place of the DNA encoding Rep protein and capsid protein is prepared. On the other hand, the DNA encoding Rep protein and capsid protein necessary for forming virus particles are inserted into other plasmids. Furthermore, a plasmid comprising genes (E1A, E1B, E2A, VA and E4orf6) responsible for the helper action of adenovirus necessary for proliferation of AAV is prepared as an adenovirus helper plasmid. The co-transfection of these three kinds of plasmids into the host cell causes the production of recombinant AAV (i.e., AAV vector) in the cell. As the host cell, a cell capable of supplying a part of the gene products (proteins) of the genes responsible for the aforementioned helper action (e.g., 293 cell, etc.) is preferably used. When such cell is used, it is not necessary to carry the gene encoding a protein that can be supplied from the host cell in the aforementioned adenoviral helper plasmid. The produced AAV vector is present in the nucleus. Thus, a desired AAV vector is prepared by destroying the host cell with freeze-thawing, collecting the virus and then subjecting the virus fraction to separation and purification by density gradient ultracentrifugation method using cesium chloride, column method or the like.

AAV vector has great advantages in terms of safety, gene transduction efficiency and the like, and is used for gene therapy. However, it is known that the size of a polynucleotide that can be packaged in AAV vector is limited. For example, in one embodiment of the present invention, the entire length including the base length of a polynucleotide comprising a base sequence encoding a fusion protein of dSaCas9 and miniVR or microVR, a base sequence encoding gRNA targeting the expression regulatory region of the human DUX4 gene, and EFS promoter sequence or CK8 promoter sequence and U6 promoter sequence as the promoter sequences, and ITR parts is about 4.85 kb, and they can be packaged in a single AAV vector.

3. Pharmaceutical Composition

The present invention also provides a pharmaceutical composition comprising the polynucleotide of the present invention or the vector of the present invention (hereinafter sometimes referred to as “the pharmaceutical composition of the present invention”). The pharmaceutical composition of the present invention can be used for treating or preventing FSHD.

The pharmaceutical composition of the present invention comprises the polynucleotide of the present invention or the vector of the present invention as an active ingredient, and may be prepared as a formulation comprising such active ingredient (i.e., the polynucleotide of the present invention or the vector of the present invention) and, generally, a pharmaceutically acceptable carrier.

The pharmaceutical composition of the present invention is administered parenterally, and may be administered topically or systemically. The pharmaceutical composition of the present invention can be administered by, but are not limited to, for example, intravenous administration, intraarterial administration, subcutaneous administration, intraperitoneal administration, or intramuscular administration.

The dose of the pharmaceutical composition of the present invention to a subject is not particularly limited as long as it is an effective amount for the treatment and/or prevention. It may be appropriately optimized according to the active ingredient, dosage form, age and body weight of the subject, administration schedule, administration method and the like.

In one embodiment of the present invention, the pharmaceutical composition of the present invention can be not only administered to the subject affected with FSHD but also prophylactically administered to subjects who may develop FSHD in the future based on the genetic background analysis and the like. The term “treatment” in the present specification also includes remission of disease, in addition to the cure of diseases. In addition, the term “prevention” may also include delaying the onset of disease, in addition to prophylaxis of the onset of disease. The pharmaceutical composition of the present invention can also be referred to as “the agent of the present invention” or the like.

4. Method for Treatment or Prevention of FSHD

The present invention also provides a method for treating or preventing FSHD, comprising administering the polynucleotide of the present invention or the vector of the present invention to a subject in need thereof (hereinafter sometimes referred to as “the method of the present invention”). In addition, the present invention includes the polynucleotide of the present invention or the vector of the present invention for use in the treatment or prevention FSHD. Furthermore, the present invention includes use of the polynucleotide of the present invention or the vector of the present invention in the manufacture of a pharmaceutical composition for the treatment or prevention of FSHD.

The method of the present invention can be practiced by administering the aforementioned pharmaceutical composition of the present invention to a subject affected with FSHD, and the dose, administration route, subject and the like are the same as those mentioned above.

Measurement of the symptoms may be performed before the start of the treatment using the method of the present invention and at any timing after the treatment to determine the response of the subject to the treatment.

5. Ribonucleoprotein

The present invention provides a ribonucleoprotein comprising the following (hereinafter sometimes referred to as “RNP of the present invention”):
(c) a fusion protein of a nuclease-deficient CRISPR effector protein and a transcription repressor, and
(d) a guide RNA targeting a continuous region set forth in SEQ ID NO: 2, 3, 4, 8, 15, 17, 18, 20, 25, 31, 32, 33, 35, 39, 40, 42, 44, 50, 51, 52, 55, 57, 58, 59, 65, 67, 68, 113, 116, 135, 138, 142, 144, 146, 156, 158, 161, or 171, preferably are selected from a base sequence comprising a sequence set forth in SEQ ID NO: 2, 3, 4, 20, 51, 68, 138, 142, 146, 156, 158, or 161 in the expression regulatory region of human DUX4 gene.

As the nuclease-deficient CRISPR effector protein, transcription repressor, and guide RNA comprised in the RNP of the present invention, the nuclease-deficient CRISPR effector protein, transcription repressor, and guide RNA explained in detail in the above-mentioned section of “1. Polynucleotide” can be used. The fusion protein of nuclease-deficient CRISPR effector protein and transcription repressor to be comprised in the RNP of the present invention can be produced by, for example, introducing a polynucleotide encoding the fusion protein into the cell, bacterium, or other organism to allow for the expression, or an in vitro translation system by using the polynucleotide. In addition, guide RNA comprised in the RNP of the present invention can be produced by, for example, chemical synthesis or an in vitro transcription system by using a polynucleotide encoding the guide RNA. The thus-prepared fusion protein and guide RNA are mixed to prepare the RNP of the present invention. Where necessary, other substances such as gold particles may be mixed. To directly deliver the RNP of the present invention to the target cell, tissue and the like, the RNP may be encapsulated in a lipid nanoparticle (LNP) by a known method. The RNP of the present invention can be introduced into the target cell, tissue and the like by a known method. For example, Lee K., et al., Nat Biomed Eng. 2017; 1:889-901, WO 2016/153012, which are incorporated herein by reference in their entireties, and the like can be referred to for encapsulation in LNP and introduction method.

In one embodiment of the present invention, the guide RNA comprised in RNP of the present invention targets continuous 18 to 24 nucleotides in length, preferably 20 to 23 nucleotides in length, more preferably 21 to 23 nucleotides in length, in the regions of “190,065,500-190,068,500” and “190,047,000-190,052,000” existing in the GRCh38/hg38 of human chromosome 4 (Chr 4).

In one embodiment of the present invention, the guide RNA comprised in RNP of the present invention targets continuous 18 to 24 nucleotides in length, preferably 20 to 23 nucleotides in length, more preferably 21 to 23 nucleotides in length, in the regions of “190,065,000-190,093,000” and “190,173,000-190,176,000” existing in the GRCh38/hg38 of human chromosome 4 (Chr 4).

6. Others

The present invention also provides a composition or kit comprising the following for suppression of the expression of the human DUX4 gene:
(e) a fusion protein of a nuclease-deficient CRISPR effector protein and a transcription repressor, or a polynucleotide encoding the fusion protein, and
(f) a guide RNA targeting a continuous region set forth in SEQ ID NO: 2, 3, 4, 8, 15, 17, 18, 20, 25, 31, 32, 33, 35, 39, 40, 42, 44, 50, 51, 52, 55, 57, 58, 59, 65, 67, 68, 113, 116, 135, 138, 142, 144, 146, 156, 158, 161, or 171, preferably are selected from a base sequence comprising a sequence set forth in SEQ ID NO: 2, 3, 4, 20, 51, 68, 113, 116, 135, 138, 142, 144, 146, 156, 158, 161, or 171 in the expression regulatory region of human DUX4 gene, or a polynucleotide encoding the guide RNA.

The present invention also provides a method for treating or preventing FSHD, comprising administering the following (e) and (f):
(e) a fusion protein of a nuclease-deficient CRISPR effector protein and a transcription repressor, or a polynucleotide encoding the fusion protein, and
(f) a guide RNA targeting a continuous region set forth in SEQ ID NO: 2, 3, 4, 8, 15, 17, 18, 20, 25, 31, 32, 33, 35, 39, 40, 42, 44, 50, 51, 52, 55, 57, 58, 59, 65, 67, 68, 113, 116, 135, 138, 142, 144, 146, 156, 158, 161, or 171, preferably are selected from a base sequence comprising a sequence set forth in SEQ ID NO: 2, 3, 4, 20, 51, 68, 113, 116, 135, 138, 142, 144, 146, 156, 158, 161, or 171 in the expression regulatory region of human DUX4, or a polynucleotide encoding the guide RNA.

The present invention also provides use of the following (e) and (f):
(e) a fusion protein of a nuclease-deficient CRISPR effector protein and a transcription repressor, or a polynucleotide encoding the fusion protein, and
(f) a guide RNA targeting a continuous region set forth in SEQ ID NO: 2, 3, 4, 8, 15, 17, 18, 20, 25, 31, 32, 33, 35, 39, 40, 42, 44, 50, 51, 52, 55, 57, 58, 59, 65, 67, 68, 113, 116, 135, 138, 142, 144, 146, 156, 158, 161, or 171, preferably are selected from a base sequence comprising a sequence set forth in SEQ ID NO: 2, 3, 4, 20, 51, 68, 113, 116, 135, 138, 142, 144, 146, 156, 158, 161, or 171 in the expression regulatory region of human DUX4 gene, or a polynucleotide encoding the guide RNA, in the manufacture of a pharmaceutical composition for the treatment or prevention of FSHD.

As the nuclease-deficient CRISPR effector protein, transcription repressor, guide RNA, as well as polynucleotides encoding them and vectors in which they are carried in these inventions, those explained in detail in the above-mentioned sections of “1. Polynucleotide”, “2. Vector” and “5. Ribonucleoprotein” can be used. The dose, administration route, subject, formulation and the like of the above-mentioned (e) and (f) are the same as those explained in the section of “3. Pharmaceutical composition”.

Other features of the invention will become apparent in the course of the following descriptions of exemplary embodiments which are given for illustration of the invention and are not intended to be limiting thereof.

### EXAMPLES

The examples describe the use of a fusion protein of dCas9 with a transcriptional repressor to suppress gene expression, in the defined expression regulatory region of human DUX4 gene that leads to the selective suppression of human DUX4 gene expression. The example also describes the definition of a specific genomic region that confers selective suppression of the human DUX4 gene without minimally affecting the expression of other genes. The method of the present invention to suppress human DUX4 gene expression represents a novel therapeutic or preventive strategy for the FSHD as described and illustrated herein.

(1) Experimental Methods

Selection of DUX4 Targeting Sequences-1

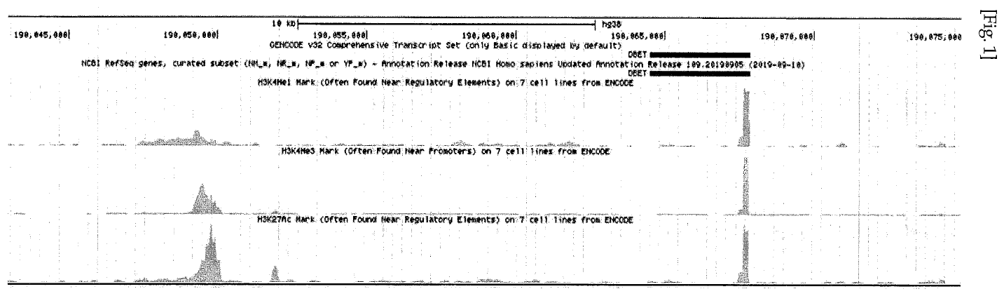

Based on the H3K4me3, H3K27Ac pattern of genome in human skeletal muscle cells, roughly 8 kb of sequence around the putative promoter regions of the human DUX4 gene was scanned for sequences that can be targeted by a catalytically-inactive SaCas9 (D10A and N580A mutant; dSaCas9) complexed with gRNA, defined herein as a targeting sequence. Location of the targeted genome regions relative to DUX4 gene is depicted in FIG. 1, and their coordinates are noted below:
1. Chr4: GRCh38/hg38; 190,065,500-190,068,500->˜3 kb (Promoter A)
2. Chr4: GRCh38/hg38; 190,047,000-190,052,000->˜5 kb (Promoter B)

Targeting sequences were specified by the 21-nucleotide segment adjacent to a protospacer adjacent motif (PAM) having the sequence NNGRRT (5′-21nt targeting sequence-NNGRRT-3′) (Tables 1-1 to 1-3).

TABLE 1-1

| SEQ ID | | | | | |
|---|---|---|---|---|---|
| No. | | Position | Strand | Sequence | PAM |
| 77 | Control-1 | N/A | N/A | ACGGAGGCTAAGCGTCGCAA | N/A |
| 78 | Control-2 | N/A | N/A | CGCTTCCGCGGCCCGTTCAA | N/A |
| 79 | Control-3 | N/A | N/A | GTAGGCGCGCCGCTCTCTAC | N/A |
| 1 | sgDUX4-1 | 190065622 | −1 | GGGAGGTGGAGCTGCCCCGGCT | TGGGGT |
| 2 | sqDUX4-2 | 190065752 | −1 | CTCATCCAGCAGCAGGCCGCAG | GGGAGT |
| 3 | sqDUX4-3 | 190065880 | −1 | AGCCCGGTATTCTTCCTCGCTG | AGGGGT |
| 4 | sgDUX4-4 | 190066039 | 1 | CCCTCCACCGGGCTGACCGGCC | TGGGAT |
| 5 | sgDUX4-5 | 190066171 | 1 | GTGGGCCGCCTACTGCGCACGC | GCGGGT |
| 6 | sqDUX4-6 | 190066333 | −1 | GGGGCCCGGTGTTTCGCGGGAC | GGGGGT |
| 7 | sqDUX4-7 | 190066493 | −1 | GCGTCCCGGTGTGCGCCGGGCC | TGGGGT |
| 8 | sgDUX4-8 | 190066576 | 1 | AACGGGAGACCTAGAGGGGCGG | AAGGAT |
| 9 | sgDUX4-9 | 190066735 | 1 | GGAAAAGCGGTCCTCGGCCTCC | GGGAGT |
| 10 | SqDUX4-10 | 190066906 | −1 | CGGGTGAAAACCCGACGGCAAC | CCGAST |
| 11 | sqDOX4-11 | 190066994 | −1 | CCTGCGTGTGGCTCCTCCGTGG | CCGGGT |
| 12 | sgDUX4-12 | 190067146 | −1 | TTGCACCCTTCCCTGCATGTTT | CCGGGT |
| 13 | sqDUX4-13 | 190067284 | 1 | CGCCGGGGAGGCATCTCCTCTC | TGGGGT |
| 14 | sgDUX4-14 | 190067420 | −1 | GAACTGAACCTCCGTGGGAGTC | TTGAGT |
| 15 | sgDUX4-15 | 190067516 | 1 | AGAGAGCGGCTTCCCGTTCCCG | CGGGAT |
| 16 | sgDUX4-16 | 190067532 | −1 | GGCCGGCTCTCCGGACCTCTCC | AGGGAT |
| 17 | SQDUX4-17 | 190067677 | −1 | GTCGAGGCCTGGGGCCGGCCGG | CGGGGT |
| 18 | sqDUX4-18 | 190067696 | 1 | GCCGGCCCCAGGCCTCGACGCC | CTGGGT |
| 19 | sgDUX4-19 | 190067708 | 1 | CCTCGACGCCCTGGGTCCCTTC | CGGGGT |

TABLE 1-2

| | | | | | |
|---|---|---|---|---|---|
| 20 | sgDUX4-20 | 190067753 | 1 | GGGGGCTCACCGCCATTCATGA | AGGGGT |
| 21 | sqDUX4-21 | 190067757 | 1 | GGCAGGCAGGCTCCACCCCTTC | ATGAAT |
| 22 | sgDUX4-22 | 190067922 | −1 | GGCCATCGCGGTGAGCCCCGGC | CGGAAT |
| 23 | sgDUX4-23 | 190067958 | 1 | TTCCGCGGGGAGGGTGCTGTCC | GAGGGT |
| 24 | sqDUX4-24 | 190067971 | −1 | CTCGTCCCCGGGCTTCCGCGGG | GAGGGT |
| 25 | sgDUX4-25 | 190068012 | −1 | CTCGCTTTGGCTCGGGGTCCAA | ACGAGT |
| 26 | sgDUX4-26 | 190068114 | 1 | AGGCCATCGGCATTCCGGAGCC | CAGGGT |
| 27 | sgDUX4-27 | 190068236 | 1 | CGGCGAAAGCGGACCGCCGTCA | CCGGAT |
| 28 | sqDUX4-28 | 190068339 | 1 | GAGAGACGGGCCTCCCGGAGTC | CAGGAT |
| 29 | sqDUX4-29 | 190068439 | 1 | CCTGTGCAGCGCGGCCCCCGGC | GGGGGT |
| 30 | sgDUX4-30 | 190068482 | 1 | TGGGTCGCCTTCGCCCACACCG | GCGAGT |
| 31 | sgDUX4-31 | 190048092 | −1 | TTTCAGTTTCCCTTTACTTGCA | GTGAAT |
| 32 | sgDOX4-32 | 190048802 | −1 | TCCAAAATGCAACCTGAAGCTA | CTGAGT |
| 33 | sqDUX4-33 | 190048960 | −1 | CACTTTCAATGGCGCCTTTTTC | ACGAAT |
| 34 | sgDUX4-34 | 190049067 | 1 | GGAAGATTTCTCTCAAAAACGG | TTGAGT |
| 35 | sgDUX4-35 | 190049179 | −1 | GAGGAGTACGTCTTCTGCAGCC | CAGGGT |
| 36 | sgDUX4-36 | 190049317 | −1 | CAGGTCTGGGCTGAGCATTGAG | GAGGGT |
| 37 | sgDUX4-37 | 190049456 | 1 | CCTCCCTCCAGGGCTATGGACC | CCGGAT |
| 38 | sqDUX4-38 | 190049553 | 1 | AGCTCCCAGCATATTAGGTGGG | GCGGGT |
| 39 | sgDUX4-39 | 190049650 | 1 | GTAGCCCTTTGACCAAAGGGTT | GGGAGT |
| 40 | SQDUX4-40 | 190049789 | 1 | GCAGCCCTGTGACCAAAGGGCT | GGGAGT |
| 41 | sgDUX4-41 | 190049881 | −1 | CCCAGCATGCACCAGGCTCAGG | GAGAGT |
| 42 | SqDUX4-42 | 190050023 | −1 | COCAGCATGCACCAGGCTCAGG | GAGAGT |
| 43 | sqDUX4-43 | 190050155 | 1 | GAAGCTCAGTTGTGTTCACTTT | TTGGAT |
| 44 | sgDUX4-44 | 190050250 | −1 | AACTATAGCTATCTGCAATATC | ATGAAT |
| 45 | sgDUX4-45 | 190050823 | −1 | ATACGATCCAGCAATTTCACAT | CTGGGT |
| 46 | sqDUX4-46 | 190047610 | −1 | GTCAAGCAGATGCCAAGTCGT | AGGGAT |
| 47 | sgDUX4-47 | 190047312 | −1 | TCAGTAAATGTTCTGTCATTG | CAGGAT |
| 48 | sgDUX4-48 | 190047373 | 1 | ATGAGCCAAGTGAAAACACCA | AGGGGT |

TABLE 1-3

| | | | | | |
|---|---|---|---|---|---|
| 49 | sgDUX4-49 | 190047553 | −1 | GGCATCACAAATGGCAAACCA | GTGAAT |
| 50 | sgDUX4-50 | 190047704 | 1 | AATGCATAAAGCCTGAAGGGA | CAGGGT |
| 51 | sqDUX4-51 | 190047832 | 1 | GAACGGACAGCAAACGACAAG | CTGAGT |
| 52 | sgDUX4-52 | 190047916 | −1 | ATCGATAAATACGCTAACCAC | ATGGGT |
| 53 | sgDUX4-53 | 190047980 | −1 | GTCACCCGCATTCTTTAGAGA | GAGAAT |
| 54 | sgDUX4-54 | 190048279 | 1 | GAATATTTTGAAAATTAAGTG | CAGAAT |
| 55 | sgDUX4-55 | 190048373 | 1 | TGCTGGAGAAACCCTCTTTAT | GGGAGT |
| 56 | sgDUX4-56 | 190048520 | 1 | TAGCATOTTAAATCTCCACCC | AGGAAT |
| 57 | sqDUX4-57 | 190048596 | 1 | AATCAAAATGCACATGCTCTG | TAGAAT |
| 58 | sgDUX4-58 | 190048649 | −1 | ACTTTTAAGACGTGGAGCACT | TGGCGT |
| 59 | sgDUX4-59 | 190048832 | 1 | TTTTGGAGCTTGUCCACTTAA | GTGGGT |
| 60 | sqDUX4-60 | 190048874 | 1 | ACAAGACTCACGCCTCAGAAA | TGGGGT |
| 61 | sgDUX4~61 | 190049206 | 1 | CGTACTCCTCTGGAAAGAACC | CTGGGT |
| 62 | sgDUX4-62 | 190049296 | 1 | GGAAAAGGTAAGGTATCTTTG | TAGGGT |
| 63 | sgDDX4-63 | 190049365 | −1 | TGCCGACTCTTTGCAAGGAGA | GTGAGT |
| 64 | sgDUX4-64 | 190049472 | −1 | AGCTATAATCCCAGCATGTAC | CGGGAT |
| 65 | sgDOX4-65 | 190049574 | −1 | GCGCAGCCCTGGAAGAAGGGG | CAGAGT |
| 66 | sgDUX4-66 | 190049662 | 1 | CAAAGGGTTGGGAGTGTTTAT | GAGAAT |
| 67 | sqDUX4-67 | 190050399 | −1 | GCAAGTTGAAAGAAGGTCCCT | ATGAGT |
| 68 | sgDUX4-68 | 190050465 | −1 | GTAGTTOTTAGGAGCTAGAGG | GGGAGT |
| 69 | sgDUX4-69 | 190050521 | −1 | TCATTCCACTTATGAGATACT | TAGAGT |
| 70 | SqDUX4-70 | 190050615 | 1 | CATGCATGACGTACCATGTGT | CAGAAT |
| 71 | sgDUX4-71 | 190050709 | 1 | TGATGGTAATTCAAACAACAC | TTGAGT |
| 72 | sqDUX4-72 | 190050923 | 1 | ACATTTCCACCAACAGTGTAC | AAGGAT |
| 73 | sqDUX4-73 | 190051026 | 1 | GGTATTAAGTGATATGTCATT | TGGGGT |
| 74 | sqDUX4-74 | 190051168 | 1 | AAGTAGTTTGTTTTATTGTTG | CTGAAT |
| 75 | sgDUX4-75 | 190051280 | −1 | TAGGGGAAACACGTCATAACA | CTGGAT |
| 76 | sqDUX4-76 | 190051357 | −1 | CTGTAAGAAAAAATTAACTAA | CTGGAT |

Selection of DUX4 Targeting Sequences-2

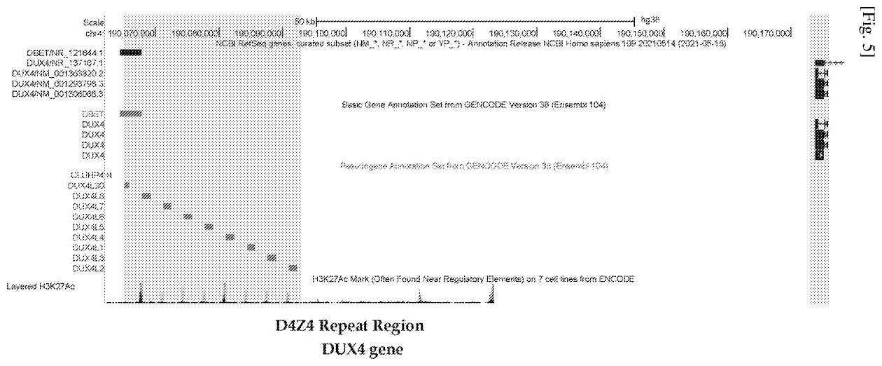

In a previous selection (Selection of DUX4 Targeting Sequences-1) we tested targeting sequences in roughly 8 kb of sequence around the putative promoter regions of the human DUX4 gene, based on the H3K4me3, H3K27Ac pattern of genome in human skeletal muscle cells. We now expanded our focus on a larger area that included the entirely of the D4Z4 repeat region and the DUX4 coding gene. The regions were scanned for sequences that can be targeted by a catalytically-inactive SaCas9 (D10A and N580A mutant; dSaCas9) complexed with gRNA, defined herein as a targeting sequence. Locations of the targeted genome regions relative to the DUX4 gene are depicted in FIG. 5, and their coordinates are noted below:
1. Chr4: GRCh38/hg38; 190,065,000-190,093,000→˜28 kb (D4Z4 repeat region)
2. Chr4: GRCh38/hg38; chr4:190,173,000-190,176,000→˜3 kb (DUX4 gene)

Targeting sequences were specified by the 21-nucleotide segment adjacent to a protospacer adjacent motif (PAM) having the sequence NNGRRT (5′-21nt targeting sequence-NNGRRT-3′) (Tables 2-1 to 2-4).

TABLE 2-1

| SEQ ID | | | | | |
|---|---|---|---|---|---|
| No. | | Position | Strand | Sequence | PAM |
| 77 | Control-1 | N/A | N/A | ACGGAGGCTAAGCGTCGCAA | N/A |
| 78 | Control-2 | N/A | N/A | CGCTTCCGCGGCCCGTTCAA | N/A |
| 79 | Control-3 | N/A | N/A | GTAGGCGCGCCGCTCTCTAC | N/A |
| 2 | sqDUX4-2 | 190065752 | −1 | CTCATCCAGCAGCAGGCCGCAG | GGGAGT |
| 20 | sgDUX4-20 | 190067753 | 1 | GGGGGCTCACCGCCATTCATGA | AGGGGT |
| 51 | sgDUX4-51 | 190047832 | 1 | GAACGGACAGCAAACGACAAG | CTGAGT |
| 98 | sgDUX4-82 | 190065019 | 1 | CTAAGGCTTTTTCTCTCCCTCC | CAGAAT |
| 99 | sgDUX4-83 | 190065047 | −1 | TGGGAGGATTTTGCCTGTGAGT | TCGAAT |
| 100 | sqDUX4-84 | 190065053 | −1 | AGATTCTGGGAGGATTTTGCCT | GTGAGT |
| 101 | sqDUX4-85 | 190065061 | 1 | GAACTCACAGGCAAAATCCTCC | CAGAAT |
| 102 | 59DUX4-86 | 190065066 | −1 | TTATGTTCTCACAAGATTCTGG | GAGGAT |
| 103 | sgDUX4-87 | 190065110 | 1 | TGACTAGTTTGGCATTGCTTTT | GGGGAT |
| 104 | sgDDX4-88 | 190065156 | −1 | GATTTATAAATAATGGCATGAC | AAGGGT |
| 105 | sgDUX4-89 | 190065218 | 1 | GTGAGGGGAGATGGGGAGACAT | TGGGAT |
| 106 | sqDUX4-90 | 190065317 | 1 | CCAGCCAGGCCGCGCCGGCAGA | GGGGAT |
| 107 | sqDUX4-91 | 190065345 | 1 | CTCCCAACCTGCCCCGGCGCGC | GGGGAT |
| 108 | sgDUX4-92 | 190065408 | −1 | TGCGGAGGCCACCGAGGAGCCT | GAGGGT |
| 109 | sgDDX4-93 | 190065435 | −1 | TCCCGGTCCTCCCGGCTTTTGC | CCGGGT |
| 110 | 9gDUX4-94 | 190065462 | −1 | GGGCCCGGCAGGCCGTCGCGCT | GCGGGT |
| 111 | sqDUX4-95 | 190065526 | 1 | CTCAAGCGGGGCCGCAGGGCCA | AGGGGT |
| 112 | sgDUX4-96 | 190065553 | −1 | CCACCACGGACTCCCCTGGGAC | GTGGGT |
| 113 | sgDUX4-97 | 190065554 | 1 | GCTTGCGCCACCCACGTCCCAG | GGGAGT |
| 114 | sgDUX4-98 | 190065578 | 1 | GAGTCCGTGGTGGGGCTGGGGC | CGGGGT |
| 115 | sgDUX4-99 | 190065918 | 1 | CTGCTGGAGGAGCTTTAGGACG | CGGGGT |
| 116 | sqDUX4-100 | 190065929 | 1 | GCTTTAGGACGCGGGGTTGGGA | CGGGGT |
| 117 | SODUX4-101 | 190065934 | 1 | AGGACGCGGGGTTGGGACGGGG | TCGGGT |
| 118 | sgDUX4-102 | 190066055 | −1 | CACCGGCCTGGACCTAGAAGGC | AGGAAT |

TABLE 2-2

| | | | | | |
|---|---|---|---|---|---|
| 119 | sgDUX4-103 | 190066087 | −1 | AGAATGGCAGTTCTCCGCGGTG | TGGAGT |
| 120 | sgDUX4-104 | 190066110 | −1 | GGGATCCCCGGGATGCCCAGGA | AAGAAT |
| 121 | sgDUX4-105 | 190066117 | 1 | GCCATTCTTTCCTGGGCATCCC | GGGGAT |
| 122 | sgDUX4-106 | 190066124 | −1 | CCTGGGCCGGCTCTGGGATCCC | CGGGAT |
| 123 | sgDUX4-107 | 190066133 | −1 | CTGCTGGTACCTGGGCCGGCTC | TGGGAT |
| 124 | sgDUX4-108 | 190066254 | −1 | TGGGGATGGGGCGGTCAGGCGG | CGGGGT |
| 125 | sgDUX4-109 | 190066275 | −1 | CCGGGGGTGGGGGGTGGGGGGT | GGGGAT |
| 126 | sqDUX4-110 | 190066281 | 1 | CGTTTTCCGGGGGTGGGGGGTG | GGGGGT |
| 127 | sgDUX4-111 | 190066288 | −1 | GACGACGCGTTTTCCGGGGGTG | GGGGGT |
| 128 | sqDUX4-112 | 190066295 | 1 | CCCAGGGGACGACGCGTTTTCC | GGGGGT |
| 129 | sgDOX4-113 | 190066311 | 1 | GGAAAACGCGTCGTCCCCTGGG | CTGGGT |
| 130 | sgDUX4-114 | 190066759 | −1 | GCTGACCGTTTTCCCGGAGGGC | GGGGGT |
| 131 | sgDUX4-115 | 190066843 | −1 | CTGGGCCCCGGAACCGGGGCGA | ATGGGT |
| 132 | sgDUX4-116 | 190066847 | −1 | CTCCCTGGGCCCCGGAACCGGG | GCGAAT |
| 133 | sgDUX4-117 | 190066858 | 1 | TTCGCCCCGGTTCCGGGGCCCA | GGGAGT |
| 134 | sqDOX4-118 | 190066896 | 1 | CTCCGGGACAAAAGACCGGGAC | TCGGGT |
| 135 | sgDUX4-119 | 190066907 | 1 | AAGACCGGGACTCGGGTTGCCG | TCGGGT |
| 136 | sqDUX4-120 | 190066929 | −1 | GGATGTGCGGTCTGTGAACCGC | GOGGGT |
| 137 | sgDUX4-121 | 190066953 | −1 | GCCGCGTTGCAGGGCTCAGCCT | GGGGAT |
| 138 | sgDUX4-122 | 190067152 | 1 | GGGCACCCGGAAACATGCAGGG | AAGGGT |
| 139 | sgDUX4-123 | 190067229 | −1 | ATTCCCGCGTGCGGCAACGTGG | GGGAGT |
| 140 | sgDOX4-124 | 190067239 | 1 | ACTCCCCCACGTTGCCGCACGC | GGGAAT |
| 141 | sqDUX4-125 | 190067255 | 1 | TCCCCGGCGTGATGGCCTGACG | ATGGAT |
| 142 | sgDUX4-126 | 190067427 | −1 | GAGTGTGGAACTGAACCTCCGT | GGGAGT |
| 143 | SqDUX4-127 | 190067451 | −1 | AAACCAGCCTGGGAGGGTGGAG | GGGAGT |
| 144 | sqDUX4-128 | 190067461 | −1 | CAGCAGGGAGAAACCAGCCTGG | GAGGGT |
| 145 | sgDOX4-129 | 190068022 | −1 | CTCGCAGGGCCTCGCTTTGGCT | CGGGGT |
| 146 | sgDUX4-130 | 190068065 | −1 | TCTCTGGTGGCGATGCCCGGGT | ACGGGT |
| 147 | sqDOX4-133 | 190068071 | −1 | AGCCGTTCTCTGGTGGCGATGC | CCGGGT |
| 148 | sgDUX4-132 | 190068115 | −1 | ACCAAATCTGGACCCTGGGCTC | CGGAAT |

TABLE 2-3

| | | | | | |
|---|---|---|---|---|---|
| 149 | sgDUX4-133 | 190068133 | 1 | GCCCAGGGTCCAGATTTGGTTT | CAGAAT |
| 150 | sgDUX4-134 | 190068170 | 1 | CGCCAGCTGAGGCAGCACCGGC | GGGAAT |
| 151 | sgDUX4-135 | 190068252 | −1 | GGCTCGGAGGAGCAGGGCGGTC | TGGGAT |
| 152 | sgDUX4-136 | 190068274 | 1 | CCTGCTCCTCCGAGCCTTTGAG | AAGGAT |
| 153 | sgDUX4-137 | 190068332 | 1 | CTGGCCAGAGAGACGGGCCTCC | CGGAGT |
| 154 | sgDUX4-138 | 190068355 | −1 | CCCTTCGATTCTGAAACCAGAT | CTGAAT |
| 155 | sgDOX4-139 | 190068358 | 1 | GTCCAGGATTCAGATCTGGTTT | CAGAAT |
| 156 | sgDUX4-140 | 190068385 | 1 | TCGAAGGGCCAGGCACCCGGGA | CAGGGT |
| 157 | sgDUX4-141 | 190068389 | 1 | GCGGGCGCCCTGCCACCCTGTC | COGGGT |
| 158 | sgDOX4-142 | 190068458 | −1 | GCGAAGGCGACCCACGAGGGAG | CAGGGT |
| 159 | sgDUX4-143 | 190068459 | 1 | GCGGGGGTCACCCTGCTCCCTC | GTGGGT |
| 160 | sgDUX4-144 | 190068519 | −1 | AGCCCCAGGCGCGCAGGGCACG | TGGGGT |
| 161 | sgDUX4-145 | 190069348 | −1 | ACCGGGCCTAGACCTAGAAGGC | AGGAAT |
| 162 | sqDUX4-146 | 190069575 | 1 | GCGTTTTCCGGGGGTGGGGGGT | GGGGGT |
| 163 | sgDOX4-147 | 190069784 | −1 | CGTCCCCGGTGTGCGCCGGGCC | TGGGGT |
| 164 | sqDUX4-148 | 190070198 | −1 | CGGGTGAAGACCCGACGGCAAC | CCGAGT |
| 165 | sgDUX4-149 | 190070221 | −1 | GGATGTGGGGTCTGTGAACCGC | GCGGGT |
| 166 | sgDUX4-150 | 190070530 | 1 | GACTCCCCACGTTGCCGCACGC | GGGAAT |
| 167 | sgDUX4-151 | 190070946 | −1 | GGTGGTGGTGGTGGTGGGGGGG | GGGGGT |
| 168 | sqDUX4-152 | 190071909 | 1 | CCAGCCAGGCCGCGCCGGCAGA | GGGGGT |
| 169 | sgDUX4-153 | 190072645 | −1 | ACCGGGCCTGGACCTAGAAGGC | AGGAAT |
| 170 | sgDUX4-154 | 190072845 | −1 | TGGGGAGGGGGGGGTCAGGCGG | CGGGGT |
| 171 | sgDUX4-155 | 190073814 | 1 | GATTCCCGCGTGCGGCAACGTG | GGGAGT |
| 172 | sgDUX4-156 | 190173479 | −1 | TGGTGGTGGTGGTGGTGGGGGG | GGGGGT |
| 173 | sgDUX4-157 | 190175220 | −1 | AGAAAGGCAGTTCTCCGCGGAG | TGGAGT |
| 174 | sgDUX4-158 | 190175225 | −1 | AGGAAAGAAAGGCAGTTCTCCG | CGGAGT |
| 175 | sgDUX4-159 | 190175250 | 1 | GCCTTTCTTTCCTGGGCATCCC | GGGGAT |
| 176 | sqDUX4-160 | 190175673 | −1 | GCGAGCTCCCTTGCACGTCAGG | CGGGGT |
| 177 | sgDUX4-161 | 190175727 | 1 | TTGTTCTTCCGTGAAATTCTGG | CTGAAT |
| 178 | sgDUX4-162 | 190175732 | −1 | AAGGTGGGGGGAGACATTCAGC | CAGAAT |

TABLE 2-4

| | | | | | |
|---|---|---|---|---|---|
| 179 | sgDUX4-163 | 190175768 | 1 | TTCCGACGCTG | CTGGAT |
| | | | | TCTAGGCAAAC | |
| 180 | sgDUX4-164 | 190175774 | 1 | CGCTGTCTAGG | TAGAGT |
| | | | | CAAACCTGGAT | |
| 181 | sgDUX4-165 | 190175788 | 1 | ACCTGGATTAG | CTGGAT |
| | | | | AGTTACATCTC | |
| 182 | sgDUX4-166 | 190175830 | 1 | TATATTAAAAT | GTGGAT |
| | | | | GCCCCCTCCCT | |

Construction of Lentiviral Transfer Plasmid (pED316)

pLentiCRISPR v2 was purchased from Genscript (https://www.genscript.com) and the following modifications were made: the SpCas9 gRNA scaffold sequence was replaced by SaCas9 gRNA scaffold sequence; SpCas9-FLAG was replaced with dSaCas9 (D10A and N580A mutant) fused to the Kruppel associated box (KRAB) domain. KRAB transcriptional suppression domain can suppress gene expression when localized to promoters by recruiting suppressive elements. KRAB was tethered to the C-terminus of dSaCas9, which is referred to as dSaCas9-KRAB hereinafter, and targeted to human DUX4 gene regulatory regions as directed by targeting sequences (Tables 1 and 2). The generated backbone plasmid was named pED316.

gRNA Cloning

Three negative control non-targeting sequences, three positive control targeting sequences, 76 targeting sequences (Table 1), and 85 targeting sequence (Table 2) were cloned into pED316. Forward and reverse oligos were synthesized by Integrated DNA Technologies in the following format: Forward; 5′ CACC(G)-21 basepair targeting sequence—3′, and Reverse: 5′ AAAC—19-21 basepair reverse complement targeting sequence—(C)—3′, where bases in parenthesis were added if the target did not begin with a G. Oligos were resuspended in Tris-EDTA buffer (pH 8.0) at 100 μM. 1 μl of each complementary oligo were combined in a 10 μl reaction in NE Buffer 3.1 (New England Biolabs). The reaction was heated to 95° C. and allowed to cool to 25° C. in a thermocycler, thus annealing oligos with sticky end overhangs compatible with cloning to pED316. Annealed oligos were combined with lentiviral transfer plasmid pED316 which had been digested with BsmBI and gel purified, and ligated with T4 DNA ligase (NEB catalog number: M0202S) according to manufacturer's protocol. 2 μl of the ligation reaction was transformed into 10 μl of NEB Stable Competent cells (NEB catalog number: C3040I) according to the manufacturer's protocol. The resulting construct drives expression of sgRNAs comprising crRNA encoded by individual targeting sequences fused with tracrRNA (gttttagtactctggaaacagaatctactaaaacaaggcaaaatgccgtgtttatctcgtcaacttgttggcgagatttttt; SEQ ID NO: 97) by a U6 promoter.

Lentivirus Generation

HEK293TA cells were seeded at 0.75×10⁶ cells/well (for the targeting sequences listed in Table 1) or 1×10⁶ cells/well (for the targeting sequences listed in Table 2) in 6 well cell culture dishes (VWR catalog number: 10062-892) in 2 ml growth medium (DMEM media supplemented with 10% FBS and 2 mM fresh L-glutamine, 1 mM sodium pyruvate and non-essential amino acids) and incubated at 37° C./5% CO₂ for 24 hours. The next day TransIT-VirusGEN transfection reactions were set up according to manufacturer's protocol with 1.5 μg packaging plasmid mix [1 μg packaging plasmid (see pCMV delta R8.2; addgene #12263) and 0.5 [cg envelope expression plasmid (see pCMV-VSV-G; addgene #8454)] and 1 μg of transfer plasmid pED316 containing sequence encoding dSaCas9-KRAB and indicated sgRNAs. Lentivirus was harvested 48 hours (for the targeting sequences listed in Table 1) or 72 hours (for the targeting sequences listed in Table 2) following transfection by passing media supernatant through a 0.45 m PES filter (VWR catalog number: 10218-488). Until ready to use, the purified and aliquoted lentiviruses were stored in −80° C. freezer.

Transduction of FSHD Patient Derived Lymphoblast Cell Lines (LCLs)

When Using the Targeting Sequences Listed in Table 1:

Two FSHD patient derived B-lymphoblast cell lines (LCLs) GM16343 and GM16414 were obtained from Coriell Institute. The cells were cultured in RPMI-1640 medium supplemented with 15% fetal bovine serum. For transduction, 100,000 cells were mixed with 8 μg/ml Polybrene (Sigma catalog number: TR-1003-G) and 200 μl lentivirus supernatants (see above) corresponding to each sgRNA (Table 1) was added to each well (96-well plate). Cell and virus mixture were then spun down for 1 hour at 1200×g, 37° C., followed by resuspension in fresh media at 0.25-0.5×10⁶ cells per ml. 72 hours after transduction, cells were fed selection medium [growth media supplemented with 0.5 μg/ml puromycin (Sigma Aldrich catalog number: P8833-100MG)]. Cells were given fresh selection medium every 2-3 days. Following 7-15 days of cells being in selection medium, cells were harvested and RNA extracted with RNeasy 96 kit (Qiagen catalog number: 74182) as directed by manufacturer.

When Using the Targeting Sequences Listed in Table 2:

An FSHD patient derived B-lymphoblast cell lines (LCLs) GM16343 was obtained from Coriell Institute. The cells were cultured in RPMI-1640 medium supplemented with 15% fetal bovine serum. For transduction, 500,000 cells were mixed with 6.66 μg/ml Polybrene (Sigma catalog number: TR-1003-G) and 500 μl lentivirus supernatants (see above) corresponding to each sgRNA (Table 2) was added to each well (24-well plate). Cell and virus mixture were then spun down for 1 hour at 1200×g, 37° C., followed by resuspension in fresh media at 0.5×10⁶ cells per ml. 72 hours after transduction, cells were fed selection medium [growth media supplemented with 0.5 μg/ml puromycin (Sigma Aldrich catalog number: P8833-100MG)]. Cells were given fresh selection medium every 2-3 days. Following 14-17 days of cells being in selection medium, cells were harvested and RNA extracted with RNeasy 96 kit (Qiagen catalog number: 74182) as directed by manufacturer.

Gene Expression Analysis

For gene expression analysis, cDNA was generated from ˜0.5-0.8 μg of total RNA according to High-Capacity cDNA Reverse Transcription Kit (Applied Biosystems; ThermoFisher catalog number: 4368813) protocol in a 10 μl volume. cDNA was diluted 10-fold and analyzed using Taqman Fast Advanced Master Mix according to manufacturer's protocol. Taqman probes (DUX4: Custom designed; MBD3L2: Assay Id Hs00544743_m1; TRIM43: Assay Id Hs00299174_m1; ZSCAN4: Assay Id Hs00537549_m1; HPRT: Assay Id Hs99999909_m1 VIC_PL) were obtained from Life Technologies. Taqman probe-based real-time PCR reactions were processed and analyzed by QuantStudio 5 Real-Time PCR system as directed by Taqman Fast Advanced Master Mix protocol.

Data Analysis

For each sample and controls, deltaCt values were calculated by subtracting the average Ct values from 3 technical replicates of the target gene (DUX4, MBD3L2, TRIM43 and ZSCAN4) probe from the HPRT probe (Average Ct DUX4−Average Ct HPRT). The average deltaCt of the 3 control sgRNAs was then calculated. The deltaCt of each sample and control was then subtracted from the average control deltaCt to obtain a deltadeltaCt value for each sample and control. Normalized expression values were then determined for each sample and control using the formula 2\hat{ }-(deltadeltaCt). In this case the individual expression value of each control is normalized to the average expression value of all three control samples. The graphed bar denoting the control sample is an average of the three controls over three separate experiments.

(2) Results

Suppression of DUX4 Gene Expression by the dSaCas9-KRAB:sgRNA

In the initial sgRNA screening experiment, lentivirus was produced that deliver expression cassettes for dSaCas9-KRAB and sgRNAs for each targeting sequence to FSHD patient derived LCLs. Transduced cells were selected for resistance to puromycin for 7 days (for the targeting sequences listed in Table 1) or 14 days (for the targeting sequences listed in Table 2), and DUX4 expression was quantitated using the Taqman Assay. Expression values from each sample were normalized to an average of DUX4 expression in cells transduced with control sgRNAs.

When Using the Targeting Sequences Listed in Table 1:

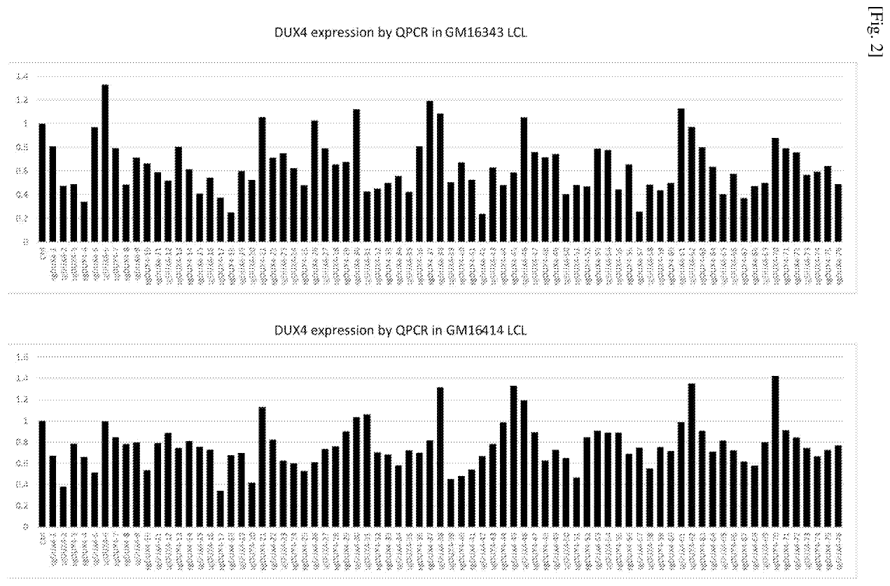

As shown in FIG. 2, out of 76 tested sequences, 27 targeting sequences showed at least 50% down-regulation of DUX4 mRNA expression in either of the two LCLs (FIG. 2 and Table 3).

TABLE 3
27 selected sgRNA treated LCLs DUX4 expression
level compared to control sgRNAs (set as 1.0)
from initial sgRNA screening experiment

| | GM16343 | GM16414 |
|---|---|---|
| sgDUX4-2 | 0.47 | 0.38 |
| sgDUX4-3 | 0.49 | 0.78 |
| sgDUX4-4 | 0.34 | 0.66 |
| sgDUX4-8 | 0.48 | 0.78 |
| sgDUX4-15 | 0.41 | 0.75 |
| sgDUX4-17 | 0.37 | 0.34 |
| sgDUX4-18 | 0.25 | 0.68 |
| sgDUX4-20 | 0.53 | 0.42 |
| sgDUX4-25 | 0.48 | 0.53 |
| SqDUX4-31 | 0.43 | 1.06 |
| sgDUX4-32 | 0.45 | 0.70 |
| sgDUX4-33 | 0.50 | 0.68 |
| sgDUX4-35 | 0.42 | 0.72 |
| sgDUX4-39 | 0.50 | 0.45 |
| sgDUX4-40 | 0.67 | 0.48 |
| sgDUX4-42 | 0.24 | 0.67 |
| sgDUX4-44 | 0.48 | 0.98 |
| sgDUX4-50 | 0.40 | 0.65 |
| sgDUX4-51 | 0.48 | 0.46 |
| sgDUX4-52 | 0.47 | 0.84 |
| sgDUX4-55 | 0.44 | 0.89 |
| sgDUX4-57 | 0.26 | 0.75 |
| sgDUX4-58 | 0.49 | 0.55 |
| sgDUX4-59 | 0.44 | 0.75 |
| sgDUX4-65 | 0.41 | 0.81 |
| sgDUX4-67 | 0.37 | 0.62 |
| sgDUX4-68 | 0.47 | 0.58 |

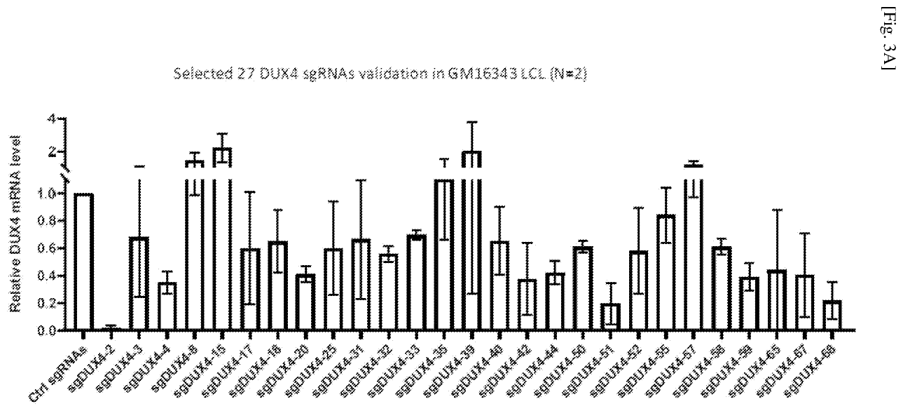

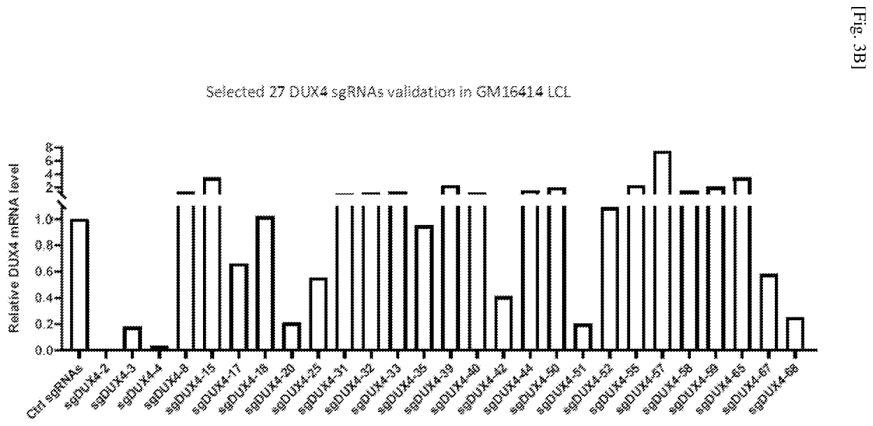

Next, we carried out a validation screening with these 27 most potent candidate sgRNAs identified from the initial screening, this time transduced cells were selected for resistance to puromycin for 15 days to explore the possibility that longer treatment will yield better suppression. As shown in FIG. 3, 7 sgRNA targeting sequences showed at least 50% down-regulation of DUX4 mRNA expression in both two LCLs, with sgRNA-#2 showing around 99% suppression. This result also suggested that for certain sgRNAs, suppression potency can be greatly increased by longer treatment.

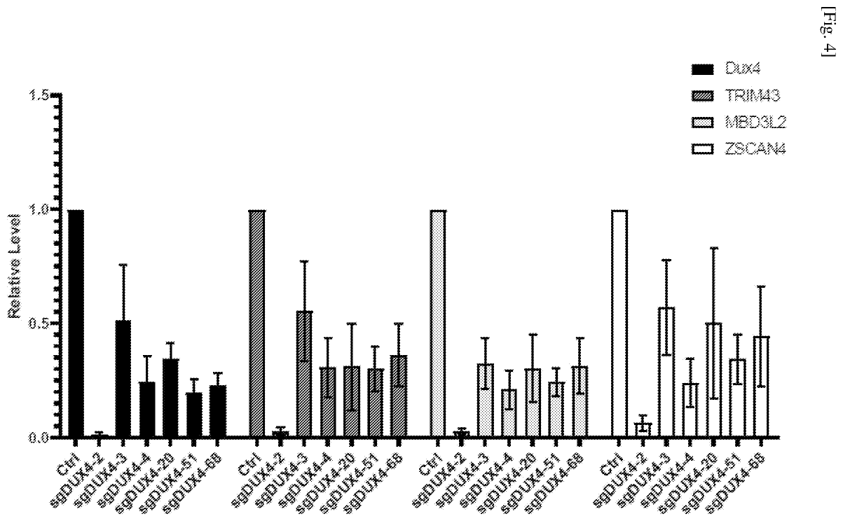

Expression of DUX4 causes the aberrant upregulation of many downstream targets, including genes expressed in the germline and in early development. TRIM43, ZSCAN4, and MBD3L2 are downstream targets of DUX4 that were also found to be upregulated in the FSHD patient derived LCLs cultures used in this study. To determine whether dCas9-KRAB-mediated repression of DUX4 also results in repression of these DUX4 target genes, we measured levels of TRIM43, ZSCAN4, and MBD3L2 from samples treated with most potent sgRNAs identified in the validation experiment. As expected, all these dCas9-KRAB:sgRNAs significantly reduced expression of all three DUX4 targets to ˜50-99% of endogenous levels (FIG. 4), and the suppression potency is strongly correlated with DUX4 suppression potency (Table 4)

TABLE 4
Pearson Correlation analysis of DUX4, TRIM43, MBD3L2
and ZSCAN4 mRNA level in best 6 sgRNAs (sgDUX4-2, 3,
4, 20, 51, and 68) treated FSHD patient derived LCLS.

| | DUX4 | TRIM43 | MBD3L2 | ZSCAN4 |
|---|---|---|---|---|
| DUX4 | 1.000 | 0.984 | 0.961 | 0.965 |
| TRIM43 | | 1.000 | 0.964 | 0.965 |
| MBD3L2 | | | 1.000 | 0.954 |
| ZSCAN4 | | | | 1.000 |

(The numbers shown in table are the Pearson correlation coefficients for each of DUX4, TRIM43, MBD3L2 and ZSCAN4 mRNA levels)

When Using the Targeting Sequences Listed in Table 2:

In addition to 85 new targeting sequences, 3 additional targeting sequences previously shown to suppress DUX4 expression (sgDUX4-2, 20, 51) were tested for comparison.

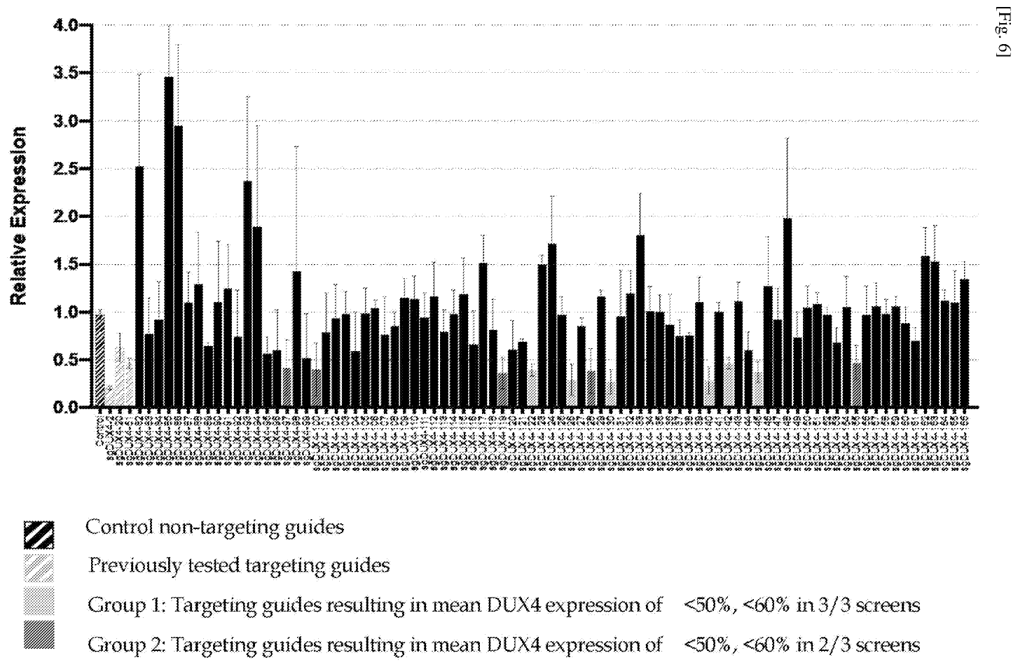

Out of 85 new targeting sequences tested, 11 targeting sequences showed a mean down-regulation of DUX4 mRNA expression of at least 50% from 3 screening experiments (FIG. 6). Of these 11 targeting sequences, 6 targeting sequences resulted in DUX4 expression of <60% compared to control non-targeting sequences in all three individual screening experiments [Group 1]. The other 5 of 11 targeting sequences resulted in DUX4 expression of <60% in 2 out of 3 individual screening experiments [Group 2] (FIG. 6, Table 5).

TABLE 5
DUX4 expression level of 11 best sgRNA treated
LCLs compared to control sgRNAs (set as 1.0)
from initial sgRNA screening experiment

| | |
|---|---|
| Group 1 | Group 2 |

| | Expression | | Expression |
|---|---|---|---|
| sgDUX4-122 | 0.3937 | sgDUX4-97 | 0.4204 |
| sgDUX4-126 | 0.2916 | sgDUX4-100 | 0.401 |
| sgDUX4-130 | 0.273 | sgDUX4-119 | 0.3611 |
| sgDUX4-140 | 0.2856 | sgDUX4-128 | 0.3877 |
| sgDUX4-142 | 0.4622 | sgDUX4-155 | 0.4719 |
| sgDUX4-145 | 0.3729 | | |

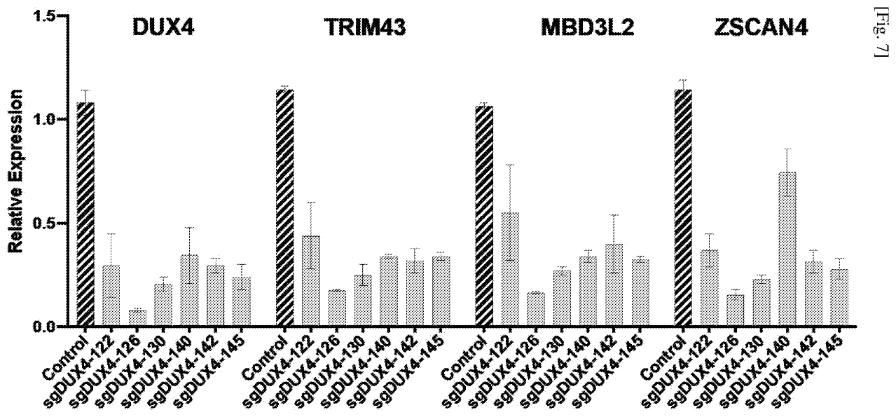

Next, we carried out a validation screening with the 6 most potent candidate sgRNAs identified from the initial screening [Group 1] this time transduced cells were selected for resistance to puromycin for 17 days to explore the possibility that longer treatment will yield better suppression (FIG. 7).

Expression of DUX4 causes the aberrant upregulation of many downstream targets, including genes expressed in the germline and in early development. TRIM43, ZSCAN4, and MBD3L2 are downstream targets of DUX4 that were also found to be upregulated in the FSHD patient derived LCLs cultures used in this study. To determine whether dCas9-KRAB-mediated repression of DUX4 also results in repression of these DUX4 target genes, we measured levels of TRIM43, ZSCAN4, and MBD3L2 from samples treated with most potent sgRNAs identified in the validation experiment. As expected, all these dCas9-KRAB:sgRNAs significantly reduced expression of all three DUX4 targets (FIG. 7), and the suppression potency is strongly correlated with DUX4 suppression potency (Table 6).

TABLE 6
Pearson Correlation analysis of DUX4, TRIM43, MBD3L2 and
ZSCAN4 mRNA level in best 6 sgRNAs (sgDUX4-122, 126, 130,
140, 142, 145) treated FSHD patient derived LCLs.

| | DUX4 | TRIM43 | MBD3L2 | ZSCAN4 |
|---|---|---|---|---|
| DUX4 | 1.000 | 0.988 | 0.964 | 0.926 |
| TRIM43 | | 1.000 | 0.983 | 0.885 |
| MBD312 | | | 1.000 | 0.849 |
| ZSCAN4 | | | | 1.000 |

(The numbers shown in table are the Pearson correlation coefficients for each of DUX4, TRIM43, MBD3L2 and ZSCAN4 mRNA levels)

### INDUSTRIAL APPLICABILITY

According to the present invention, the expression of DUX4 gene in human cells can be suppressed. Thus, the present invention is expected to be extremely useful for the treatment and/or prevention of FSHD.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is, therefore, to be understood that, within the scope of the appended claims, the invention may be practiced otherwise than as specifically described herein.

All patents and other references mentioned above are incorporated in full herein by this reference, the same as if set forth at length.

This application is based on U.S. provisional patent application No. 63/072,327 (filing date: Aug. 31, 2020), and U.S. provisional patent application No. 63/235,359 (filing date: Aug. 20, 2021), both filed in US, the contents of which are incorporated in full herein.

1. A polynucleotide, comprising the following base sequences:
(a) a base sequence encoding a fusion protein of a nuclease-deficient CRISPR effector protein and a transcription repressor, and
(b) a base sequence encoding a guide RNA targeting a continuous region set forth in SEQ ID NO: 2, 3, 4, 20, 51, 68, 138, 142, 146, 156, 158, or 161 in the expression regulatory region of human DUX4 gene.

2. The polynucleotide according to claim 1, wherein the base sequence encoding the guide RNA comprises the base sequence set forth in SEQ ID NO: 2, 3, 4, 20, 51, 68, 138, 142, 146, 156, 158, or 161 or the base sequence set forth in SEQ ID NO: 2, 3, 4, 20, 51, 68, 138, 142, 146, 156, 158, or 161 in which 1 to 3 bases are deleted, substituted, inserted, and/or added.

3. The polynucleotide according to claim 1, comprising at least two different base sequences encoding the guide RNA.

4. The polynucleotide according to claim 1, wherein the transcriptional repressor is selected from the group KRAB, MeCP2, SIN3A, HDT1, MBD2B, NIPP1, and HP1A.

5. The polynucleotide according to claim 4, wherein the transcriptional repressor is KRAB.

6. The polynucleotide according to claim 1, wherein the nuclease-deficient CRISPR effector protein is dCas9.

7. The polynucleotide according to claim 6, wherein the dCas9 is derived from Staphylococcus aureus.

8. The polynucleotide according to claim 1, further comprising a promoter sequence for the base sequence encoding the guide RNA and/or a promoter sequence for the base sequence encoding the fusion protein of the nuclease-deficient CRISPR effector protein and the transcriptional repressor.

9. The polynucleotide according to claim 8, wherein the promoter sequence for the base sequence encoding the guide RNA is selected from the group U6 promoter, SNR6 promoter, SNR52 promoter, SCR1 promoter, RPR1 promoter, U3 promoter, and H1 promoter.

10. The polynucleotide according to claim 9, wherein the promoter sequence for the base sequence encoding the guide RNA is U6 promoter.

11. The polynucleotide according to claim 8, wherein the promoter sequence for the base sequence encoding the fusion protein of the nuclease-deficient CRISPR effector protein and the transcriptional repressor is a ubiquitous promoter or a neuron specific promoter.

12. The polynucleotide according to claim 11, wherein the ubiquitous promoter is selected from the group EFS promoter, CMV promoter and CAG promoter.

13. A vector comprising a polynucleotide according to claim 1.

14. The vector according to claim 13, wherein the vector is a plasmid vector or a viral vector.

15. The vector according to claim 14, wherein the viral vector is selected from the group adeno-associated virus (AAV) vector, adenovirus vector, and lentivirus vector.

16. The vector according to claim 15, wherein the AAV vector is selected from the group AAV1, AAV2, AAV6, AAV7, AAV8, AAV9, Anc80, AAV₅₈₇MTP, AAV₅₈₈MTP, AAV-B1, AAVM41, and AAVrh74.

17. The vector according to claim 16, wherein the AAV vector is AAV9.

18-19. (canceled)

20. A method for treating or preventing FSHD, comprising administering a polynucleotide according to claim 1, or a vector comprising said polynucleotide, to a subject in need thereof.
